(19) United States
(12) Patent Application Publication
(10) Pub. No.: US 2006/0030524 A1
(43) Pub. Date: Feb. 9, 2006

(54) Treatment of systemic lupus erythematosus by down-regulating the autoimmune response to autoantigens

(75) Inventors: Irun R. Cohen, Rehovot (IL); Varda Rotter, Rishon Lezion (IL); Neta Erez, Tel-Aviv (IL); Johannes Herkel, Weisbaden (DE)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co. Ltd., Rehovot (IL)

(21) Appl. No.: 11/179,820

(22) Filed: Jul. 13, 2005

Related U.S. Application Data
(63) Continuation of application No. 09/807,827, filed on Jul. 12, 2001, now abandoned.
(60) Provisional application No. 60/104,816, filed on Oct. 19, 1998.

(51) Int. Cl. A61K 48/00; A61K 38/17

(52) U.S. Cl. 514/12; 514/44

(57) ABSTRACT

Systemic lupus erythematosus (SLE) can be prevented or treated by down-regulating the autoimmune response to the C-terminal-DNA-binding domain of the p53 protein (p53) by an active principle selected from the group consisting of: (i) a peptide of, or comprising, the C-terminal DNA-binding domain of the p53 protein; (ii) a monoclonal antibody (mAb) specific for said domain of p53 (Ab1), and fragments thereof; (iii) an mAb specific for Ab1 (hereinafter Ab2), and fragments thereof; (iv) a peptide based on a complementarity determining region (CDR) of the heavy or light chain of said Ab1 or Ab2; (v) a DNA molecule coding for (i) and (iv) of for the variable region of said Ab1 and Ab2 of (ii) and (iii); and (vi) T cells specific for (i) to (iv), fragments thereof, T cell receptor (TCR) thereof and peptides comprising the variable region of said TCR. SLE can also be diagnosed by assaying for antibodies (Ab1) against the C-terminal DNA-binding domain of p53 or antibodies (Ab2) specific to the Ab1 antibodies.

### CROSS-REFERENCE TO RELATED APPLICATIONS

This application is a continuation of U.S. application Ser. No. 09/807,827, filed Jul. 12, 2001, now abandoned, which is a 371 of PCT/US99/24443, filed Oct. 19, 1999, which claims the benefit of U.S. Provisional Application No. 60/104,816, filed Oct. 19, 1998. The entire contents of all of the above are hereby incorporated by reference.

### FIELD OF THE INVENTION

The present invention is directed to a method for preventing or treating systemic lupus erythematosus (SLE) by down-regulating the autoimmune response to the major autoantigens that maintain the autoimmune reaction. It is also directed to diagnostic methods and kits for SLE, to pharmaceutical compositions for use in such treatment and diagnosis, and to novel peptides.

### BACKGROUND OF THE INVENTION

The rejection of transplanted cells and tissues of allogeneic origin proves that the immune system is capable of destroying its targets. However, in some cases, an individual's immune system destroys itself, resulting in an autoimmune disease such as diabetes mellitus, multiple sclerosis, rheumatoid arthritis, systemic lupus erythematosus, etc.

Antibodies to DNA are characteristic of many autoimmune diseases, notably systemic lupus erythematosus (SLE) and particularly lupus nephritis. However, there is at present no generally accepted explanation for the prevalence of anti-DNA antibodies in autoimmune disorders. Immunity to DNA appears to be driven by an antigen (Radic et al, 1994), but self-DNA is unlikely to be the driving antigen because mammalian DNA usually does not induce an anti-DNA immune response (Pisetsky, 1996).

SLE is known to be associated with antibodies to various autoantigens, notably to DNA and to nuclear antigens. It is not clear, however, what drives and maintains the immune response to these antigens, and how such immunity might be involved in pathogenesis. It has been reported that certain common anti-DNA idiotypes can induce SLE in susceptible strains of mice (Mendlovic et al, 1988) and that antibodies, anti-idiotypic to these common idiotypes, play a role in lupus development (Ward et al, 1997). However, the “natural” antigens of these idiotypic antibodies have not been defined.

It is the variable region of an antibody (antigen-binding site) which binds to an antigen. Therefore, the variable regions of antibodies have three-dimensional structures that are complementary to the structures of the antigenic determinants the antibodies recognize.

The binding site of the antibody complementary to the structure of the antigen is created by hypervariable regions of the light and heavy chains of the Fab portion of the antibody. These binding site structures are formed by the collective aggregate of the complementarity determining region (CDR) of the light and heavy chains of the immunoglobulin molecule. However, an antibody itself, when recognized by another antibody, can be considered to be an antigen. In the case where structures of the variable regions of the antibody are recognized, these structures are called idiotypes, and the antibodies that recognize the idiotypes of the antibody are called anti-idiotypic antibodies. The structure corresponding to the antigenic determinant of the antibody is called an idiotope.

It has been reported that immunization with monoclonal antibodies can induce immune responses that extend beyond the specificity of the antibody, probably by anti-idiotypic connectivity based on idiotypic determinants in the variable regions of the immunizing monoclonal antibody. According to idiotypic antibody network terminology, Ab1 is the first antibody, the antibody binding to the antigen, and Ab2 is the anti-idiotypic antibody to Ab1. The variable region of Ab2 may mimic the conformation of the antigen because both the antigen and Ab2 can be bound by Ab1. Ab3 is the anti-idiotypic antibody to Ab2. Because of the chain of structural complementarity, Ab1 and Ab3 can have similar specificity for the original antigen.

Treatment of autoimmune disease has been shown to be possible by inducing immunological tolerance to the major-autoantigens that maintain the autoimmune reaction an immune response which destroys T cells which attack the autoantigens, or inducing a TH1→e TH2 shift in the T cells reactive to the autoantigens. In experimental models of multiple sclerosis, for example, tolerance to myelin basic protein (MBP) and clinical improvement of disease could be achieved in a variety of ways:

1. T-cell vaccination with attenuated anti-MBP T cells (Ben-Nun et al, 1981);

2. Vaccination with peptides derived from the T cell receptor (TCR) of anti-MBP T cells (Vandenbark et al, 1989);

3. Vaccination with DNA encoding the TCR of anti-MBP T cells (Waisman et al, 1996);

4. Intravenous or intraperitoneal application of MBP or MBP-derived peptides (Gaur et al, 1992);

5. Oral tolerance induction by enteric administration of MBP or MBP-derived peptides (Higgins et al, 1988).

Similar techniques have been disclosed in U.S. Pat. No. 5,578,303 with respect to insulin dependent diabetes mellitus (IDDM) and the 65 kD heat shock protein, which is cross-reactive with the major autoantigen of IDDM. The entire contents of U.S. Pat. No. 5,578,303 are hereby incorporated herein by reference.

The p53 protein is a tumor-associated antigen which is the product of a tumor suppressor gene that functions to arrest the growth of mutated or aberrant cells (Baker et al, 1990). Functional p53 is believed to sense DNA damage (Lee et al, 1995) and subsequently induce DNA repair (Kastan et al, 1991), growth arrest (Kuerbitz et al, 1992), or apoptosis (Yonish-Rouach et al, 1991) of the aberrant cells. The sequence of murine p53, as reported in Shohat-Foord et al (1991), is set forth in SEQ Q ID NO:1. The DNA and amino acid sequences of human p53, as reported in Harris et al (1986), are set forth in SEQ ID NOs:9 and 10.

The p53 protein has at least two DNA-binding sites:

(1) the core of the p53 protein, which interacts specifically with a DNA sequence in the promoters of p53-responsive genes (el-Deiry et al, 1992), and

(2) the C-terminus of the p53 protein, which can recognize features common to damaged DNA in general (Lee et al, 1995; Shohat-Foord et al, 1991).

The p53 protein is a transcription factor that binds specifically to a consensus site present in the regulatory sequences of p53-dependent genes (el-Deiry et al, 1992). Mutation of the p53 gene in the domain encoding binding to the specific DNA regulatory site causes a loss of tumor suppression. Therefore, it is not surprising that a significant proportion of natural human tumors bear mutated p53 (Hollstein et al, 1991).

Inactivation of the p53 tumor suppressor protein by mutation of the gene or by viral insertion, gene rearrangement, or other causes is a common event in human cancers. Point mutation or deletion of the p53 gene is the most common genetic aberration in human neoplasms. Approximately 70% of colon cancers, 30 to 50% of breast cancers, 50% of lung cancers, and almost 100% of small-cell carcinomas of the lung harbor p53 mutations (Hollstein et al, 1991). The p53 protein, both mutant and wild-type, can accumulate in the cytoplasm of cancer cells, and cancer patients have been found to produce antibody and T cell responses to p53. Normal cells express p53 to a much lower degree and, unlike tumor cells, normal cells show no accumulation of p53 in the cytoplasm. Thus, tumor cells and normal cells differ in both the amount and compartment of p53 expression. Although both mutated p53 and wild-type p53 have been used as immunogens for tumor immunotherapy, p53 is not very immunogenic, probably because it is a self-protein and, therefore, is immunologically tolerated.

In the laboratory of the present inventors, it has been observed that the immunization of BALB/c mice to an Ab1 anti-p53 antibody specific for a mutant domain of 53 could activate an idiotypic network leading to anti-mutant p53 immunity and resistance to a tumor (Ruiz et al, 1998; Erez-Alon et al, 1998). Mice were immunized with domain-specific anti-p53 monoclonal antibodies (Ab1): PAb-248 (Yewdell et al, 1986) directed to the N-terminus; PAb-246 (Yewdell et al, 1986; Cook et al, 1990) directed to the specific DNA-binding region; or PAb-240 directed to a mutant p53 that does not bind specific DNA. Immunized mice responded by making anti-idiotypic antibodies (Ab2) specific for Ab1 inducer. Ab1 PAb-246 induced Ab2 that, like p53 itself, could bind the specific DNA oligonucleotide sequence of the p53 responsive element. Mice immunized with Ab1 PAb-240 or PAb-246 spontaneously made Ab3 anti-p53 antibodies that reflected the specificity of their Ab1 inducers: Ab1 PAb-246 induced Ab3 specific for wild-type p53; PAb-240 induced Ab3 specific for mutant p53. Ab1 PAb-248 induced only Ab2. The spontaneously arising Ab3 were of T cell-dependent IgG isotypes. Peptides from the complementarity determining region of the Ab1 antibodies PAb-240 and PAb-246 could also induce Ab3 anti-p53. Finally, mice that produced Ab3 anti-p53 acquired resistance to tumor metastases. Therefore, an anti-idiotypic network built around certain domains of p53 seems to be programmed within the immune system, specific Ab2 antibodies can mimic the DNA binding domain of p53, and Ab3 network immunity to p53 can be associated with resistance to tumor cells.

### SUMMARY OF THE INVENTION

It is an object of the present invention to treat or prevent autoimmune diseases involving an autoimmune reaction against DNA, including systemic lupus erythematosus (SLE).

It is another object of the present invention to provide peptide, antibody and T cell products and compositions for use in the treatment or prevention of SLE.

It is a further object of the present invention to provide a method for diagnosis of SLE.

It is yet another object of the present invention to provide methods and kits for use in the diagnosis of SLE.

These and other objects of the present invention are attained based on the discovery that injection of BALB/c mice with a monoclonal antibody which binds the non-specific DNA binding site at the C-terminus of p53 induced SLE in the immunized mice. Furthermore, an anti-idiotypic network was induced by this monoclonal antibody in the SLE-susceptible BALB/c mice. It was, thus, surmised that anti-idiotypic antibodies, specific to the monoclonal antibodies which are specific to the C-terminal DNA-binding domain of p53, attack not only the anti-p53 monoclonal antibodies but also the DNA bound by this domain of p53, thereby inducing the symptoms of SLE. Therefore, development of SLE and of anti-DNA antibodies appears to occur as a consequence of an idiotypic immune response to the DNA binding protein p53.

Since the development of experimental SLE is now discovered to be driven by an immune response to the DNA-binding domain of the C-terminus of the p53 protein, abrogation of the immune reaction can be expected to result in prevention of SLE or, in SLE patients, clinical improvement of SLE disease.

The present invention thus relates to a method for preventing or treating systemic lupus erythematosus (SLE) in a human, comprising down-regulating the autoimmune response in the human to the C-terminal DNA-binding domain of the p53 protein (p53). Said down-regulating step comprises administering to the human, in a manner that suppresses the autoimmune response to the C-terminal DNA-binding domain of the p53 protein, an active principle selected from the group consisting of:

(i) a peptide of the C-terminal DNA-binding domain of the p53 protein of the sequence consisting of the residues-364-383 of the p53 protein (residues 364-383 of SE ID NO:1) or a peptide or polypeptide including said sequence;

(ii) a monoclonal antibody (mAb) specific for the C-terminal DNA-binding domain of the p53 protein raised against a peptide or polypeptide of (i) (hereinafter Ab1), and fragments thereof;

(iii) a mAb specific for Ab1 (hereinafter Ab2), and fragments thereof;

(iv) a peptide based on a complementarity determining region (CDR) of the heavy or light chain of said Ab1 or Ab2 of (ii) or (iii), respectively;

(v) a DNA molecule coding for (i) or (iv) or for the variable region of said Ab1 and Ab2 of (ii) and (iii); and

(vi) T cells specific for (i) to (iv), fragments thereof, T cell receptor (TCR) thereof and peptides comprising the variable region of said TCR.

In one aspect of the invention, the active principle is the T cell as defined in (vi) above. It is preferably a T cell product selected from the group consisting of:

(a) activated human T cells that manifest specificity for the C-terminal DNA-binding domain of the p53 protein, or to a monoclonal antibody specific for the C-terminal DNA-binding domain of the p53 protein (Ab1), or to a monoclonal antibody specific for Ab1 (Ab2);

(b) said human T cells of (a) which have been attenuated by gamma-or W irradiation or by pressure treatment by means of hydrostatic pressure, treatment with chemical cross-linking agent and/or treatment with a cytoskeletal cross-linking agent;

(c) fragments, or surface proteins shed from, the T cells of (a) or (b); and

(d) a T cell receptor (TCR) of the T cells of (a) or a peptide comprising the variable region of said TCR.

In a preferred embodiment of this aspect of the invention, the human T cells are ones which manifest specificity for the C-terminal DNA-binding domain of the p53 protein. In another preferred embodiment, the T cells are attenuated by gamma-or W irradiation. The T cells may be autologous T cells from the patient to be treated or semi-allogeneic T cells from a donor sharing at least one HLA class II molecule with said patient, e.g., one of the parents or a sibling.

Abrogation or down-regulation of the immune reaction which causes SLE can be attained by inducing appropriate immunological tolerance or appropriate suppression of immune response in manners which are already known per se with respect to the prevention and treatment of other autoimmune diseases in which the major autoantigen is known. Among the ways that this immune reaction can be abrogated are the following.

T cell vaccination using attenuated autoimmune T cells constitutes a preferred embodiment of the invention. It activates regulatory mechanisms without paying the price of acute disease. Anti-idiotypic T cells quell the autoimmune T cells and so prevent the clinical emergence of the disease or result in clinical improvement of the disease. With respect to SLE, the complicated immunological response which it is desired to disrupt includes the antibodies which are generated in the SLE patient against the C-terminal DNA-binding domain of the p53 protein (the Ab1 antibodies) and the Ab2 antibodies which are generated against the Ab1 antibodies. As the Ab1 antibodies mimic the DNA to which the C-terminal domain of p53 interacts, it is the Ab2 antibodies which are specific to the Ab1 antibodies and, thus, cross-reactive with the DNA, thereby initiating the autoimmune attack against DNA, causing SLE. Thus, interruption of this idiotypic network, at any point, will down-regulate the autoimmune response which causes the autoimmune anti-DNA attack which results in SLE.

Accordingly, T cell vaccination can be accomplished with T cell lines specific for the C-terminal DNA-binding domain of the p53 domain, T cells which are specific for the Ab1 antibody, or T cells which are specific for the Ab2 antibody. The T cells may be taken directly from a patient who is to be treated (autologous T cells) or may be obtained from a donor who shares at least one HLA class II molecule with the patient (semi-allogeneic T cells), e.g., from one of the parents or a sibling. These specific cells can be activated either by incubating in the presence of the antigen or by incubating with a mitogen capable of inducing an immune response by the T cells, such as Concanavalin A or phytohemagglutinin. Such activated T cells are preferably attenuated, preferably by gamma-or UV irradiation, or by a means of attenuation which also has the salutary effect of increasing the immunogenicity of the T cells, such as by pressure treatment by means of hydrostatic pressure of sufficient pressure and time to cause augmented immunogenicity of the T cells without substantial loss of membrane protein therefrom. Alternatively, the pressure may be of sufficient magnitude and duration to cause the cell surface proteins to be shed from the cells. After low speed centrifugation to remove the cells, the fragments obtained after high speed centrifugation may be used as the vaccine, as well as the soluble proteins remaining in the supernatant after high-speed centrifugation. All of these techniques are described in detail in European patent publication 261,648 of the present applicants, the entire contents of which being hereby incorporated herein by reference.

The specific, activated T cells may also be attenuated by treatment with a chemical cross-linking agent, such as formaldehyde, glutaraldehyde or a photoactivatable psoralen cross-linking agent, such as 8-methoxypsoralen (see European patent publication 333,606 to the present applicants, the entire contents of which being hereby incorporated herein by reference). Such T cells may also be treated with a cytoskeletal disrupting agent, such as cytochalsin or colchicine. Any one or more of the pressure-treatment, chemical cross-linking treatment and cytoskeletal disrupting agent treatment steps can be combined. In addition, the cells so treated may be lysed and only the fixed cell membranes recovered and used. All of these processes are described in detail in European patent publication 261,648, incorporated by reference hereinabove.

The variable region of the T cell receptor specific for the p53 C-terminal DNA-binding domain, specific to the Ab1 antibody, or specific to the Ab2 antibody, and preferably the VDJ region or the VJ region of such T cell receptor, may be isolated and, preferably, cloned for expression and used as the T cell vaccine preparation of the present invention in the manner discussed in Howell et al (1989) and Vandenbark (1989) for the autoimmune encephalomyelitis T cell receptor.

Alternatively, any of the antigens which generate antibodies or T cells within the idiotypic network which leads to the disease-causing Ab2 antibodies may be administered in such a way as to down-regulate the immune response, such as by causing a TH1-→TH2 shift or inducing anergy or otherwise inducing tolerance for the administered peptide so as to prevent an active immune attack thereagainst. Thus, the C-terminal DNA-binding domain of p53, Ab1 or at least the antigen binding domain of Ab1, or the corresponding T cell against the C-terminal DNA-binding domain of p53 (Tc1) or at least the variable region of the TCR thereof, or Ab2 or at least the antigen binding domain of Ab2, or the corresponding T cell (Tc2) or at least the variable region of the TCR thereof, may be administered in such a way as to create tolerance or anergy or otherwise to suppress immune response and cause a TH1→TH2 shift, stopping the self-destruction of DNA. The active principle should be administered in such a manner so as to induce anergy, create tolerance or otherwise cause a TH1→TH2 shift, rather than inducing a damaging immunogenic response. Thus, it should not be administered in Complete Freund's Adjuvant or other strongly immunogenic adjuvant. One way of administering the active principle such that it will induce tolerance is to administer it with a carrier that favors induction of tolerance to the antigen when the antigen-carrier conjugate is administered. Such carriers are known as tolerogenic carriers. Examples of known tolerogenic carriers are polymers of D-amino acids, polyethylene glycol, polymers of sugar molecules, self-IgG molecules, self-spleen cells, and fatty acid molecules. An antigen may also be administered in a monomeric highly-soluble form to induce tolerance. They may be administered intravenously or intraperitoneally as was described for MBP or MBP-derived peptides by Gaur et al (1992). Another known method of inducing tolerance to an antigen is to administer it orally, even without any carrier specifically chosen for its tolerogenic characteristics, as was described, for example, for MBP or MBP-derived peptides by Higgins et al (1988).

A preferred method of administering such active principle is in conjunction with a metabolizable lipid emulsion, such as INTRALIPID or LIPOFUNDIN, which promotes a TH1→TH2 cytokine shift. The use of such emulsions is described in detail in WO 97/02016, the entire contents of which being hereby incorporated herein by reference. Additionally, the efficacy of the treatment in accordance with the present invention can be detected and monitored by measuring for a TH1→TH2 T cell response shift in the manner described in detail in WO 97/02052, the entire contents of which being hereby incorporated herein by reference.

In another aspect of the invention, the active principle is a peptide of the C-terminal DNA-binding domain of the p53 protein of the sequence consisting of the residues 364-383 of the p53 protein or a peptide or polypeptide including said sequence. In one embodiment, the peptide is the peptide consisting of the residues 364-383 of the p53 protein (residues 364-383 of SEQ ID NO:1). In another embodiment, the polypeptide is the p53 protein.

In further aspects of the invention, the active principle is a monoclonal antibody selected from an Ab1 mAb specific for a polypeptide including the C-terminal DNA-binding domain of the p53 protein and an Ab2 mAb specific for a said Ab1 mAb.

A preferred Ab1 antibody in accordance with the present invention is the monoclonal antibody known as PAb-421 (Arai et al, 1986). This is an antibody against the C-terminal DNA-binding domain of murine p53 and was the antibody used in the tests of the present invention which proved induction of SLE in BALB/c mice, which tests are described in detail hereinbelow. The sequences of the variable heavy (V_{H}) and variable light (V_{L}) chains of the anti-p53 PAb-421 have been elucidated (see WO 98/56416) and as shown in FIG.- 9 herein. The sequence for the heavy chain is SEQ ID NO:2 and that for the light chain is SEQ ID NO:3. The CDRs are underlined in FIG. 9. Of course, other monoclonal antibodies can be raised against the C-terminal DNA-binding domain of p53 and used in the methods of the present invention in a manner similar to that described herein for PAb-421. The V_{H} and V_{L} sequences of any such other antibodies can readily be determined by techniques known in the art, as well as the sequences of the complementarity determining regions (CDRs). Any such monoclonal antibody can be tested for its operability in the present invention by testing to determine if it binds to the C-terminal DNA-binding domain of p53 or to peptides derived from the C-terminal DNA-binding domain of p53. If it does, it falls within the definition of an Ab1 antibody in accordance with the present invention, i.e., an antibody which recognizes the C-terminal DNA-binding domain of p53.

Similarly, Ab2 monoclonal antibodies can readily be raised using the Ab1 antibody, or the antigen binding site thereof, as an immunogen in the same manner as disclosed herein for the production of Ab1 monoclonal antibody. Preferred Ab2 monoclonal antibodies prepared according to the present invention are those designated herein as IDI-1 and IDI-2. The sequences of the VH and VL chains of IDI-1 and IDI-2 have been elucidated according to the present invention and are given in FIG. 9 and in SEQ ID NOs:4-7, respectively. The CDRs are underlined in FIG. 9. Of course, other monoclonal antibodies can be raised against the antigen binding site of an Ab1 antibody and used in the methods of the present invention in a manner similar to that described herein for IDI-1 and IDI-2. The V_{H} and V_{L} sequences of any such other antibodies can readily be determined by techniques known in the art, as well as the sequences of the CDRs. Any such monoclonal antibody can be tested for its operability in the present invention by testing to determine if it binds to an Ab1 antibody and to DNA. If it does, it falls within the definition of an Ab2 antibody in accordance with the present invention.

In still another aspect of the invention, the active principle is a peptide based on a CDR of the heavy or light chain of an Ab1 mAb raised against a polypeptide, including the C-terminal DNA-binding domain of the p53 protein, such as the anti-p53 mAb PAb-421. Based on the sequences of the heavy and light chains of PAb-421 described in PCT Publication Wo 98/56416 and shown herein in FIG. 9, and in which the CDRs are underlined, the peptides of FIG. 10, herein designated PAb-421 H1 (residues 20-39 of SEQ ID NO:2), PAb-421 H2 (residues 48-67 of SEQ ID NO:2), PAb-421 H3 (residues 93-111 of SEQ ID NO:2), PAb-421 LI (residues 22-41 of SEQ ID NO:3), PAb-421 L2 (residues 49-67 of SEQ ID NO:3), and PAb-421 L3 (residues 89-108 of SEQ ID NO:3), have been synthesized. From these peptides, PAb-421 L3 has been described in WO 98/56416 and the others are novel.

In still a further aspect of the invention, the active principle is a peptide based on a CDR of the heavy or light chain of an Ab2 mAb specific for an Ab1 mAb. The sequences of the heavy and light chains of the preferred anti-PAb-421 Ab2 mAb IDI-1 and IDI-2 are depicted in FIG. 9, in which the CDRs are underlined. Based on these sequences, the following novel peptides herein designated IDI-1 H1 (residues 22-41 of SEQ ID NO:4), IDI-1 H2 (residues 51-70 of SEQ ID NO:4), IDI-1 H3 (residues 97-115 of SEQ ID NO:4), IDI-1 LI (residues 25-44 of SEQ ID NO:5), IDI-1 L2 (residues 52-70 of SEQ ID NO:5), and IDI-1 L3 (residues 92-110 of SEQ ID NO:5), IDI-2 H1 (residues 19-38 of SEQ ID NO:6), IDI-2 H2 (residues 49-67 of SEQ ID NO:6), IDI-2 H3 (residues 96-115 of SEQ ID NO:6), IDI-2 LI (residues 11-29 of SEQ ID NO:7), IDI-2 L2 (residues 37-55 of SEQ ID NO:7), and IDI-2 L3 (residues 77-95 of SEQ ID NO:7), have been synthesized. These peptides and peptides obtained by extension or conservative amino acid substitution, and chemical derivatives of the foregoing, are encompassed by the present invention.

A “chemical derivative” of a peptide of the present invention, as defined herein, contains additional chemical moieties not normally a part of the peptide. Covalent modifications of the peptides are included within the scope of the invention. Such modifications may be introduced into the molecule by reacting targeted amino acid residues of the peptide with an organic derivatizing agent that is capable of reacting with selected side chains or terminal residues. Such derivatives include, but are not limited to, esters, N-acyl derivatives, and the like. Many such chemical derivatives and methods of making them are well known in the art.

Also included in the scope of the invention are salts, both organic and inorganic, of the CDR-based peptides.

The down-regulation of the immune response can be obtained not only by vaccinating attenuated T cells, but also by vaccinating with the TCR or peptides derived from the T cell receptor of any such T cells. Similarly, oligonucleotides may be obtained corresponding to the rearranged T cell receptor genes of any of the cloned T cells which can be used for vaccination as described above. These oligonucleotides, encoding the TCR or parts thereof, may be cloned into suitable vectors and inoculated into patients so as to produce the desired peptides in vivo, such as is described in Waisman et al (1996), the entire contents of which being hereby incorporated herein by reference.

In still another aspect of the invention, the active principle is a DNA molecule encoding the peptide or polypeptide of the active principle and said DNA is administered in a manner in which said DNA is caused to express said active principle in vivo.

Another aspect of the present invention is in the area of diagnosis of SLE. If a patient shows the presence of Ab1 antibodies against the C-terminal DNA-binding domain of p53 or Ab2 antibodies against the Ab1 antibodies, there is reason to believe that the patient either has SLE or that an outbreak of the symptoms of SLE are imminent. Accordingly, such antibodies serve as a diagnostic marker for the presence or incipience of SLE. For example, it is important to monitor SLE patients who are in remission to obtain an early indication that reemergence of SLE symptoms may be imminent. This can be accomplished with the diagnostic techniques described herein.

Thus, the present invention relates to a method for diagnosing the presence or incipience of systemic lupus erythematosus (SLE) in a patient comprising testing said patient for the presence of antibodies (Ab1) against the C-terminal DNA-binding domain of the p53 protein or antibodies (Ab2) against the Ab1 antibodies, whereby a result indicating the positive presence of either said Ab1 or Ab2 antibodies indicates a high probability of the presence or incipience of SLE.

In another embodiment, the method for diagnosing for the presence or incipience of systemic lupus erythematosus (SLE) in a patient comprises testing said patient for the presence of antibodies or T cells which immunoreact with the C-terminal DNA-binding domain of the p53 protein, or for antibodies or T cells which immunoreact with antibodies or T cells which are specific to the C-terminal DNA-binding domain of p53, whereby a result indicating the positive presence of such antibodies or T cells indicates a high probability of the presence or incipience of SLE.

One can assay for the presence of such diagnostic markers in the blood or urine being assayed by conventional techniques. For the in vitro serological tests, serum of a patient is contacted with Ab2 antibody to test for the presence of Ab1 antibody or contacted with Ab1 antibody to test for the presence of Ab2 antibody. Ab3 antibody raised against the Ab2 antibody may also be used in a serological test for the presence of Ab2 antibody. If the serum contains the antibodies being assayed for, an immunological reaction will occur, which may be detected and assayed by means of standard techniques, such as ELISA, agglutination, etc.

Any well-known immunoassay technique can be used to detect the presence of Ab1 or Ab2 antibodies or the corresponding T cells. It should be understood that once one of ordinary skill in the art becomes aware of the fact that the presence of Ab1 or Ab2 antibodies in the serum of a person, determined, for example, by means of an assay of antibodies thereagainst, is a positive indication of incipient or existing SLE, such artisans would be well aware of the types of immunoassay technique which can be used to determine whether such antibodies are present. Besides radioimmunoassay (solid or liquid phase), any conventional immunoassay technique can be used, such as enzyme-linked immunosorbent assay (ELISA), heterogeneous immunoassay (both competitive and non-competitive) using labels other than enzymes and radioisotopes, homogeneous immunoassays based on fluorescence quenching and enzyme channeling, immune precipitation (including radial immune diffusion) and agglutination assays based on visual semi-quantitative detection or quantitative turbidimetric detection. The assay may use any conventional solid phase or sandwich assay techniques. The present invention is intended to comprehend all known means of immunodetection and labeling, e.g., enzyme, fluorescent, chemiluminescent, bioluminescent or radioactive, as are well known in the art. Such techniques are known, for example, from Harlow et al, Antibodies: A Laboratory Manual, Cold Spring Harbor Laboratory (1988), Current Protocols in Immunology, eds. Coligan et al, Wiley & Sons, Inc. (1992-1996), and many other sources well known to those of ordinary skill in the art.

Similarly, kits may be prepared for carrying out any of the various assays used for accomplishing the present invention, such as kits comprising: (i) a peptide consisting of, or including, the C-terminal DNA-binding domain of the p53 protein, or an Ab1 mAb raised against said peptide or an Ab2 mAb specific for said Ab1 mAb; and (ii) a tagged antibody capable of recognizing the non-variable region of a human antibody. Each such kit would include all of the materials necessary to conduct a single assay or a fixed number of assays. For example, such a kit for determining the presence of Ab1 antibodies may contain solid-phase immobilized Ab2 antibodies and a tagged antibody capable of recognizing the non-variable region of the Ab1 antibody to be detected, such as tagged anti-human Fab. A kit for determining the presence of Ab2 antibodies may contain solid-phase immobilized Ab1 or Ab3 antibody which reacts or cross-reacts with Ab2, and a tagged antibody capable of recognizing the non-variable region of the Ab2 antibody to be detected, such as tagged anti-human Fab. The kit should also contain reagent capable of precipitating immune complexes of Ab1 or Ab2 and anti-Ab1 or Ab2 antibodies and may contain directions for using the kit and containers to hold the materials of the kit. Any conventional tag or label may be used, such as a radioisotope, an enzyme, a chromophore or a fluorophore. A typical radioisotope is iodine-125 or sulfur-35. Typical enzymes for the purpose include horseradish peroxidase, a-galactosidase and alkaline phosphatase.

Diagnostic compositions according to the present invention are prepared by combining Ab1 or Ab2 antibodies with suitable adjuvants and auxiliary components.

Another manner of conducting a diagnostic test is to inject Ab1 or Ab2 antibodies subcutaneously into a patient and to look for the occurrence of a detectable skin reaction. In the in vivo skin test, the skin reaction at the site of the injection is measured after a sufficient time period, for example, 24 to 72 hours after administration. Swelling and/or redness is due to a delayed hypersensitivity-like reaction.

The present invention further includes the novel monoclonal antibodies IDI-1 and IDI-2 and pharmaceutical and diagnostic peptides and compositions usable in the methods of the present invention.

Similarly, isolated T cells corresponding to Ab1 and Ab2, i.e., Tc1 and Tc2, are novel and part of the present invention, as are peptides made from the TCR's thereof. Such substances, as well as pharmaceutical compositions to be administered for down-regulating the immune response which causes SLE are also part of the present invention.

### BRIEF DESCRIPTION OF THE DRAWINGS

FIG. 1 is a graph showing induction of Ab2 antibodies by immunization to Ab1 anti-p53 antibodies. BALB/c mice (white bars) and C57BL/6 mice (black bars) were immunized with Ab1 PAb-246, PAb-248 or PAb-421, as indicated, and their Ab2 response to the (Fab)₂ fragments of the Ab1 antibodies were detected. BALB/c mice made anti-idiotypic Ab2 antibodies with restricted specificity to their Ab1 inducers (P<0.001). C57BL/6 mice, in response to Ab1 PAb-246 or PAb-421, made Ab2 anti-idiotypes that were cross-reactive with both these Ab1 (P<0.001). In response to Ab1

FIG. 2 is a graph showing cross-reactivity of C57BL/6 mouse Ab2 to PAb-246 and PAb-421. The Ab2 cross-reactivity between PAb-246 and PAb-421 of immunized C57BL/6 mice could be absorbed out significantly (P<0.002) by preincubation with PAb-246 (striped bars) or PAb-421 (white bars). Preincubation with PAb-248 (black bars) did not absorb out the cross-reactivity. The mean percent inhibition (+SD) of the reactivity to PAb-246 or PAb-421 by preincubation is shown.

FIG. 3 is a graph showing Ab2 antibodies to single-stranded calf thymus DNA. Sera of BALB/c and C57BL/6 mice immunized to Ab1 PAb-246, PAb-248 or PAb-421 were tested for Ab2 antibodies to single-stranded DNA from calf thymus. BALB/c mice made significant titers of Ab2 anti-DNA, when immunized to Ab1 PAb-246 (P<0.002 when compared to PAb-248 or normal serum) or to PAb-421 (P<0.001). C57BL/6 mice immunized with either Ab1, in contrast, did not make a significant anti-DNA response, when compared to normal sera.

FIGS. 4A and 4B are graphs showing the induction of Ab3 anti-p53 antibodies by immunization to Ab1 anti-p53 antibodies. Sera of BALB/c mice and C57BL/6 mice immunized to the indicated Ab1 anti-p53 antibodies or normal sera were examined for their Ab3 antibodies reactive with p53. Ab3 anti-p53 antibodies were detected with an IgG-specific secondary antibody (FIG. 4A) or with secondary antibodies specific for the IgG1, IgG2A, IgG2B or IgG3 isotopes (FIG. 4B). The specific induction of Ab3 by immunization with Ab1 PAb-246 or PAb-421 in both strains was statistically significant (P<0.0001) when compared to normal sera. PAb-248 did not significantly induce anti-p53 antibodies.

FIG. 5 is a graph showing induction of Ab3 anti-p53 antibodies reacting with the peptide epitope of PAb-421. Sera of BALB/c and C57BL/6 mice immunized to Ab1 PAb-246, PAb-248 or PAb-421 and normal sera were tested for antibodies that bind to a p53-derived peptide (364-383 of SEQ ID NO:1), which is the antigenic epitope of PAb-421. Only BALB/c mice immunized with PAb-421 made antibodies to the peptide epitope of PAb-421 (P<0.0001). In contrast, C57BL/6 mice made no detectable antibodies to this peptide.

FIG. 6 is a graph showing spontaneous increase of anti-PAb-421 and anti-p53 antibodies in MRL/MpJ-Fas^{lpr} mice. At the age of 9 and 19 weeks, sera of MRL/MpJ-Fas^{lpr} mice that develop SLE disease spontaneously were tested for antibodies that bind to p53, to a p53-derived peptide, which is the antigenic epitope of PAb-421, or to different anti-p53 monoclonal antibodies (PAb-421, PAb-248, PAb-246, PAb-240). The MRL/MpJ-Fas^{lpr} mice, spontaneously on their way to SLE disease, developed rising tiers of antibodies reactive with p53, the p53 peptide epitope of PAb-421 and PAb-421 (P<0.0001). In contrast, they did not develop significant reactivity to the other anti-p53 antibodies PAb-248, PAb-246 and PAb-240.

FIG. 7 is a graph showing the reactivities of monoclonal antibodies IDI-1 and IDI-2, which were selected for idiotypic binding to PAb-421. Neither IDI-1 nor IDI-2 showed binding to mouse monoclonal antibodies other than PAb-421 (Control mAb). The binding to single-stranded or double-stranded calf thymus DNA was assessed by ELISA after the DNA had been gamma-irradiated with the indicated dosages (0-10,000 rad).

FIG. 8 is a graph showing the reactivity of human sera from SLE patients and healthy humans to antibodies to p53, to PAb-421 and to a control antibody, R73, that does not bind p53.

FIG. 9 shows the sequences of the heavy chain and light chain variable regions of the monoclonal antibodies PAb-421 (SEQ ID NOs:2 and 3, respectively), IDI-1 (SEQ ID NOs:4 and 5, respectively) and IDI-2 (SEQ ID NOs:6 and 7, respectively). The CDRs are underlined.

FIG. 10 shows the sequences of synthetic peptides comprising the CDRs of the heavy and light chains of PAb-421, IDI-1 and IDI-2. The SEQ ID NOs for each are as follows:

PAb-421 H1: residues 20-39 of SEQ ID NO:2
H2: residues 48-67 of SEQ ID NO:2
H3: residues 93-111 of SEQ ID NO:2

PAb-421 LI: residues 22-41 of SEQ ID NO:3
L2: residues 49-67 of SEQ ID NO:3
L3: residues 89-108 of SEQ ID NO:3

IDI-1 H1: residues 22-41 of SEQ ID NO:4
H2: residues 51-70 of SEQ ID NO:4
H3: residues 97-115 of SEQ ID NO:4

IDI-1 LI: residues 25-44 of SEQ ID NO:5
L2: residues 52-70 of SEQ ID NO:5
L3: residues 92-110 of SEQ ID NO:5

IDI-2 Hi: residues 19-38 of SEQ ID NO:6
H2: residues 49-67 of SEQ ID NO:6
H3: residues 96-115 of SEQ ID NO:6

IDI-2 LI: residues 11-29 of SEQ ID NO:7
L2: residues 37-55 of SEQ ID NO:7
L3: residues 77-95 of SEQ ID NO:7

### DETAILED DESCRIPTION OF THE INVENTION

Treatment of autoimmune diseases driven by an immune response to p53, such as SLE, can be effected by down-regulating the autoimmune response to the C-terminus of the p53 protein and/or to antibodies in its idiotypic network. Abrogation of this immune reaction will result in prevention of SLE or clinical improvement of SLE.

The p53 molecule has two attributes of immunological interest: (1) because p53 binds DNA, immunity to p53 may lead to anti-DNA antibodies by an anti-id network; antibodies to a DNA-binding site of p53 can mimic DNA and, therefore, such anti-p53 antibodies might induce anti-DNA antibodies as anti-idiotypes; and (2) because p53 accumulates in transformed cells, immunity to p53 may have an anti-tumor effect.

The generation of antibodies to DNA has historically been difficult because the DNA molecule is poorly immunogenic. In particular, it would be desirable to obtain antibodies to specific DNA sequences, as such antibodies can be used to detect the presence of such sequences for purposes of diagnosing whether an individual has a specific gene or promoter sequence. A specific antibody would have an advantage over currently used PCR techniques because antibody binding, unlike the polymerase chain reaction, is easily quantifiable and needs no primers of enzymatic replication. In particular, sequence-specific anti-DNA antibodies can be used in diagnostics, for example, in detecting critical sequences in the breeding of animals and plants, in the identification of bacteria and other parasites, in determining paternity and maternity, in forensic medicine, and perhaps even to generically identify damaged DNA. Specific anti-DNA antibodies also can be useful in the isolation of specific genes for DNA vaccination, gene cloning, and gene sequencing. Antibodies to specific sequences of DNA might also be useful in activating or inhibiting particular genes for therapeutic purposes in plants, animals or humans. It has been shown that antibodies penetrate into living cells, and anti-DNA antibodies might be able to exert effects within living cells. However, DNA in general, and certainly specific sequences of mammalian DNA, are not immunogenic.

Immunization of BALB/c mice to an Ab1 anti-p53 antibody specific for a mutant domain of p53 was found to activate an idiotypic network leading to anti-mutant p53 immunity and resistance to a tumor (Ruiz et al, 1998; Erez-Alon et al, 1998). By immunizing mice with Ab1 antibodies to three other domains of p53, it was found that Ab1 antibodies to either of the DNA-binding domains of p53 can induce anti-p53 anti-idiotypic antibodies in both BALB/c and C57BL/6 mice. Only the Ab1 to the C-terminal DNA-binding domain (PAb-421) induced SLE, and only in the BALB/c mice. The specificities of the anti-idiotypic network induced by PAb-421 differed in the SLE-susceptible BALB/c and resistant C57BL/6 strains. MRL/MpJ-FdS^{lpr} mice (Andrews et al 1978), as they develop SLE disease spontaneously, make rising titers of anti-p53 antibodies and of Ab2 specific for PAb-421, but no Ab2 specific for other anti-p53 antibodies.

While the PAb-421 antibody against the C-terminal DNA-binding domain of p53 was raised against the C-terminal DNA-binding domain of murine p53, it should be understood that the C-terminus of the murine and the human p53 are both recognized by PAb-421. Indeed, it can be seen that there is an 11 amino acid residue sequence which is identical in the C-terminal DNA-binding domain of murine and human p53. See amino acids 369-379 of SEQ ID NO:1 and amino acids 372-382 of SEQ ID NO:10. This is why in Example 7 hereinbelow, for example, the murine antibodies were used to assess for human anti-p53.

While antibodies against murine p53 were used in the examples which follow, those of ordinary skill in the art will well understand that antibodies against equivalent regions of the human p53 protein, whose entire sequence is known and available (SEQ ID NO:10), can readily be raised without undue experimentation and used for the purposes of the present invention.

When using CDR peptides, either as direct therapeutics or as immunogens to raise antiidiotype antibodies, the minimum number of amino acid residues to be used is 6-9. While 6 residues fit an antibody combining site, 9 are needed for T cell recognition as the MHC binding motifs for peptides need 9 amino acids. To make a synthetic peptide from the CDR region of the V_{H} or V_{L} sequences of an Ab1, Ab2, or Ab3 antibody, one should use 6-9 residues from within the CDR region. Longer peptides can also be used, including 1-10 residues from the natural sequence on either or both flanking sites of the core 6-9 residue region, preferably to include all of the residues of the CDR region and at least 3 residues on either flank.

While the antibodies used for purposes of the present invention may be intact antibodies, preferably human monoclonal antibodies, it should be understood that it is the epitope binding site of the antibody which provides the desired function. Thus, besides the intact antibody, proteolytic fragments thereof such as the Fab or F(ab′)₂ fragments can be used. Furthermore, the DNA encoding the variable region of the antibody can be inserted into other antibodies to produce chimeric antibodies (see, for example, U.S. Pat. No. 4,816,567) or into T-cell receptors to produce T-cells with the same broad specificity (see Eshhar et al, 1990; Gross et al, 1989). Single chain antibodies can also be produced and used. Single chain antibodies can be single chain composite polypeptides having antigen binding capabilities and comprising a pair of amino acid sequences homologous or analogous to the variable regions of an immunoglobulin light and heavy chain (linked V_{H}-V_{L} or single chain Fv). Both V_{H} and V_{L} may copy natural monoclonal antibody sequences or one or both of the chains may comprise a CDR-FR construct of the type described in U.S. Pat. No. 5,091,513 (the entire contents of which are hereby incorporated herein by reference). The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are hereby incorporated herein by reference.

When used in the present specification and claims, the recitation “a molecule including the antigen-binding portion of an antibody” is intended to include not only intact immunoglobulin molecules of any isotype and generated by any animal cell line or microorganism, but also the reactive fraction thereof including, but not limited to, the Fab fragment, the Fab′ fragment, the F(ab′)₂ fragment, the variable portion of the heavy and/or light chains thereof, and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic or diagnostic labeling) moieties by means of a portion of the molecule containing such a reactive fraction.

In the pharmaceutical compositions of the present invention, the dosage of the active ingredient and the pharmaceutically acceptable excipient or carrier in the pharmaceutical composition can be readily determined by those of skill in the art.

### EXAMPLE 1

### Induction of SLE

### Materials and Methods

Mice and Antibodies

Female mice of the BALB/c or C57BL/6 strains were obtained from the animal breeding facilities at the Weizmann Institute of Science, Rehovot, Israel, and used at the age of 8-10 weeks. Mice were immunized with anti-p53 antibodies PAb-246, PAb-248, or PAb-421, which were purified from ascitic fluid by Protein A affinity chromatography (Sigma, Rehovot, Israel). Fifty micrograms of antibody emulsified in complete Freund's adjuvant were injected into the hind footpads. The mice were boosted twice at three-week intervals, subcutaneously in the flank with 20 micrograms of the antibodies in incomplete Freund's adjuvant. Sera were obtained ten days after the first boost. For detection of anti-histone antibodies, mice were bled again two weeks after a second boost. Female MRL/MpJ-Fas^{lpr} mice were obtained from Jackson, at the age of six weeks and bled twice, at the ages of 9 and 19 weeks.

Recombinant p53 and p53 Peptide Epitope of PAb-421

E. coli BL21 (DE3) cells were transformed with the T7 expression vector containing mouse p53 DNA (Shohat-Foord et al, 1991). P53 was purified as described (Wolkowicz et al, 1995). All peptides, including the p53 peptide epitope of PAb-421 (SYLKTKKGQSTSRHKKTMVK) (residues 364-383 of SEQ ID NO:1) were prepared using an automated synthesizer (Abimed AMS 422; Langenfeld, Germany) according to the manufacturer. Peptide purity was tested by analytical reverse phase HPLC and mass spectroscopic analysis.

ELISA

ELISA assays were conducted in 96-well Maxisorp plates (Nunc, Roskilde, Denmark), which were coated with 10 μg test antigen per ml in PBS. After washing and blocking with 1% BSA in PBS for one hour at 37° C., diluted test sera (0.1 ml per well) was added for one hour at 37° C., followed by one hour incubation with goat anti-mouse IgG Fc specific, or IgG-isotype specific secondary antibodies conjugated to alkaline phosphatase, diluted 1:5000 (Jackson, Philadelphia, Pa.). A substrate solution containing 0.6 mg/ml of p-nitrophenylphosphate (Sigma, Rehovot, Israel) in diethanolamine-H₂O, pH 9.8, was added, and the plates were read at 405 nm.

For anti-DNA ELISA, a secondary antibody conjugated to horseradish peroxidase, and as substrate 1 mg/ml ABTS in 0.2 M citric acid, 0.2 M Na₂HP0₄, and 3% H₂0₂ (all from Sigma) was used. The test antigens used were (Fab) 2 fragments of Ab2 anti-PAb-421, which were prepared as described (Harlow et al, 1988), recombinant p53, the p53 peptide epitope of PAb-421, histones or, after pre-coating with methylated BSA, calf thymus DNA (all from Sigma). All antibody titers are shown as the O. D. produced by the test sera at a dilution of 1:100. Dots represent individual sera, bars represent the median of each group.

Band Shift Assay

The band shift assay was performed as described (Wolkowicz et al, 1995) with the p53-responsive element oligonucleotide TCGAGAGGCATGTCTAGGCATGTCTC (SEQ ID NO: 8) (Deiry et al, 1992). Briefly, 10-20 fmol of DNA was mixed with 4 microliters of test sera, two micrograms (2 microliters) of poly di dC, and a half reaction volume (8 microliters) of buffer (25 mM Tris-HCl, 100 mM KCl, 6.25 mM EDTA, 1 mM DTT, and 10% glycerol), incubated for 15 minutes on ice and for 15 minutes at room temperature. The reaction products were separated on 4% PAGE in a 0.4% TBE running buffer.

Crithidia Luciliae Assay

Slides with Crithidia were purchased from Immco (Buffalo, N.Y.) and incubated with the test sera according to the manufacturer's protocol.

Histology

Paraffin sections of tissue samples were either stained with hematoxylin and eosin, or immunostained with a peroxidase-labeled anti-mouse-IgG antibody (Jackson, Philadelphia, Pa.) and DAB (Sigma) according to the manufacturer's instructions.

Detection of Proteinuria and Leukopenia

Proteinuria was detected with Albustix strips (Bayer, Slough, UK). Leukocytes were counted from heparinized blood following 10-fold dilution in 1% acetic acid.

Statistics

The differences between experimental groups were tested for significance with the non-parametric Mann/Whitney U test.

### Induction of SLE-Like Diseases by Ab1 Immunization

BALB/c and C57BL/6 mice were immunized with Ab1 PAb-246 or PAb-421 and were observed for the development of lupus. Mice of either strain immunized with Ab1 PAb-246 did not develop any sign of the disease. In contrast thereto, BALB/c mice immunized with Ab1 PAb-421 were found to develop a lupus-like disease. Of the 80 BALB/c mice immunized with Ab1 PAb-421, about 95% exhibited proteinuria, ranging from 0.3 g/l to 3 g/l. Histologic examination of the kidneys showed protein deposition in tubuli and pathological changes in the glomeruli, marked by thickening of the glomerular basement membrane, “wire loop” formation, endothelial proliferation, neutrophilic exudation, deposition of hyaline material in the glomerular capillaries, and nuclear degeneration of cells. Seven out of ten BALB/c mice were found to be leukopenic (<4000 leukocytes per ml) and lymphopenic (<1200 lymphocyte per ml). About 30% of the mice immunized with PAb-421 developed significant titers of anti-histone antibodies, indicating that the immune response could spread to DNA-binding molecules other than p53.

In contrast to BALB/c mice, C57BL/6 mice immunized with Ab1 PAb-421 did not develop disease. Thus, it appears that, in a susceptible genetic background, an immune response to an Ab1 antibody specific for the C-terminal DNA-binding site of p53 can induce lupus.

### Strain Differences in Ab2 Specificities

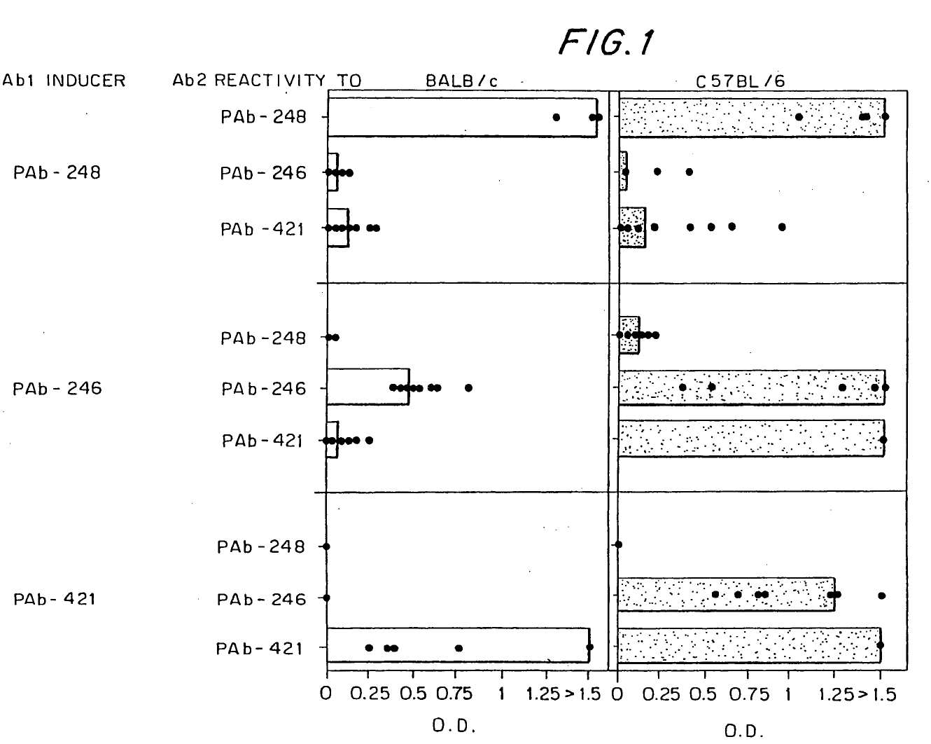

To learn whether both strains of mice responded to the Ab1 immunization by making Ab2 anti-idiotypic antibodies, the reactivity of their sera to the (Fab)₂ fragment of the Ab1 anti-p53 antibodies was determined. PAb-248 (Yewdell et al, 1986), which binds a p53 domain that does not appear to recognize DNA, was used as a control. As can be seen in FIG. 1, the BALB/c mice manifested strict Ab2 specificity: those immunized to PAb-246 made specific antibodies to PAb-246; those immunized to PAb-248 made specific antibodies to PAb-248; and those immunized to PAb-421 made antibodies specific to PAb-421.

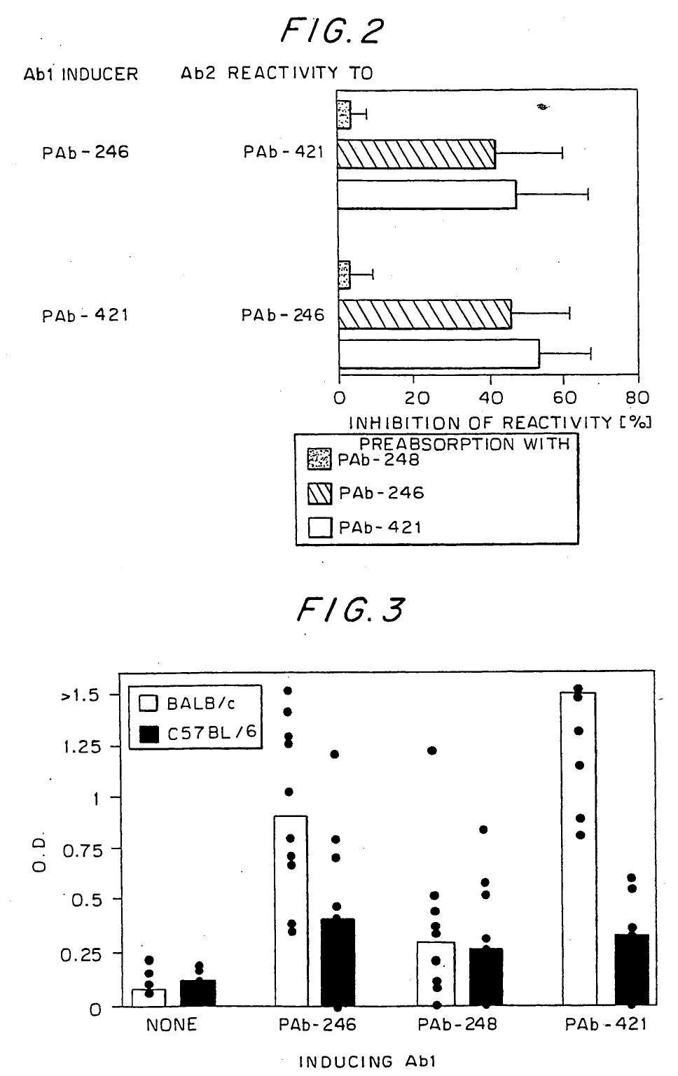

C57BL/6 mice, in contrast, made Ab2 antibodies that apparently cross-reacted to both PAb-246 and PAb-421 when immunized with PAb-426 or with PAb-421 (FIG. 1). The Ab2 cross-reactivity of the C57BL/6 mice seemed to be limited to PAb-246 and PAb-421. To confirm the Ab2 cross-reactivity between PAb-246 and PAb-421, tests were conducted to determine whether preincubation with either of the anti-p53 antibodies could absorb out the cross-reactivity. As shown in FIG. 2, the Ab2 cross-reactivity between PAb-246 and PAb-421 of immunized C57BL/6 mice could be absorbed out significantly (P<0.002) by preincubation with PAb-246 (striped bars) or PAb-421 (white bars). Preincubation with PAb-246 (black bars) did not absorb out the cross-reactivity. The mean percent inhibition (+SD) of the reactivity to PAb-246 or PAb-421 by preincubation is shown.

### Different Ab1 Antibodies Induce Different Ab2 Anti-DNA Antibodies

Because Ab1 and PAb-421 interact with p53 domains that bind different types of DNA, it was possible that the induced Ab2 antibodies might, like p53, bind to different types of DNA. To analyze the specificity of the Ab2 anti-DNA antibodies, test sera for binding to DNA were examined in three different assay systems.

It had earlier been observed that Ab2 antisera of BALB/c mice that had been induced by Ab1 PAb-246 bound specifically to the oligonucleotide DNA sequence of the p53-responsive element in a band-shift assay. That observation was reconfirmed in a band-shift assay using BALB/c sera induced by immunization to PAb-246, PAb-248 or PAb-421. C57BL/6 sera induced by immunization to each of the Ab1 antibodies were also subjected to the band-shift assay. The results of this assay showed that BALB/c mice immunized to PAb-246 produced the band shift, i.e., anti-DNA Ab2 antibodies were detected. However, BALB/c mice immunized to PAb-421 or to PAb-248 did not make such anti-DNA antibodies. In contrast to the BALB/c mice, the Ab2 antibodies of C57BL/6 mice immunized to any of the Ab1 antibodies did not recognize the p53-responsive element.

Test sera were then examined for binding to DNA using the Crithidia luciliae assay, which detects antibodies—to native, histone-free DNA exposed at the base of the Crithidia flagellum. Both BALB/c and C57BL/6 mice immunized to PAb-426 or PAb-248 were negative for anti-DNA antibodies in the Crithidia assay, as were C57BL/6 mice immunized to PAb-421. In contrast, BALB/c mice immunized to PAb-421 produced anti-DNA antibodies detectable by the Crithidia assay. The finding of positive reactivity in the Crithidia assay correlates with the clinical disease seen in the BALB/c mice. The Crithidia luciliae assay is used clinically for its diagnostic specificity for lupus in humans (Aarden et al, 1975). Crithidia luciliae is a protozoic organism and does not have a p53 gene. Thus, it is likely that its DNA does not contain p53-responsive elements, which explains why Ab1 PAb-246 did not induce anti-DNA which was detectable in the Crithidia assay.

Next, test sera were examined for binding to single stranded calf thymus DNA shown in FIG. 3. High anti-DNA reactivity could be detected in BALB/c mice immunized with PAb-246 or with PAb-421. The majority of the C57BL/6 mice immunized to either anti-p53 Ab1, in contrast, showed no significant anti-DNA reactivity. Only a minority of C57BL/6 mice showed elevated anti-DNA reactivity, mostly those that had been immunized to Ab1 PAb-246.

Thus, Ab1 antibodies to the two different DNA-binding domains of p53 both induced Ab2 anti-DNA antibodies in BALB/c mice. Ab1 PAb-246 induced anti-DNA immunity that was specific for the p53-responsive element, and, in contrast, Ab1 PAb-421 induced anti-DNA antibodies to a type associated with lupus. The anti-DNA response of the C57BL/6 mice was relatively weak.

### Generation of Ab3 Anti-p53 Antibodies

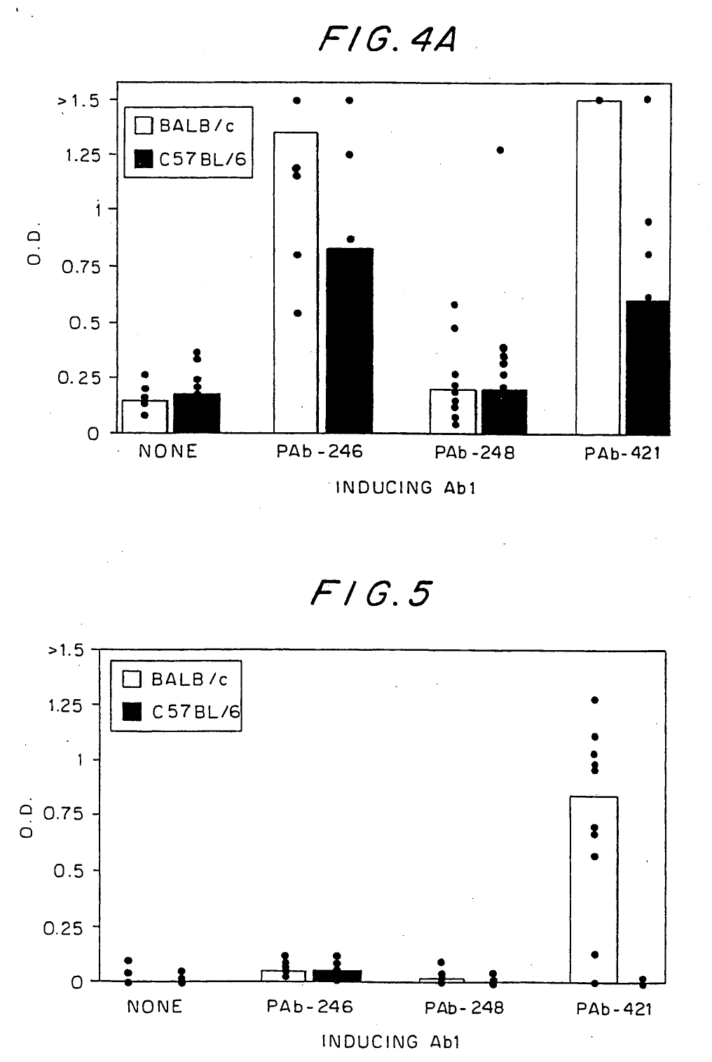

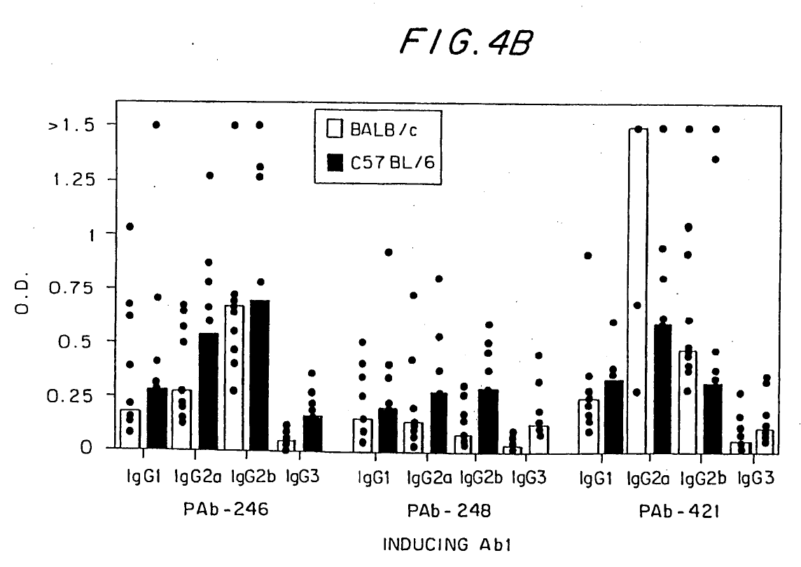

To test whether immunization to Ab1 could induce Ab3, immunized mice were assayed for antibodies to p53. Both BALB/c mice and C57BL/6 mice were found to make anti-p53 antibodies in their idiotypic network responses to PAb-421 and PAb-246 (FIG. 4A). However, Ab1 antibodies PAb-246 and PAb-421 induced a stronger Ab3 anti-p53 response in the BALB/c strain than they did in the C57BL/6 strain. Immunization to Ab1 PAb-248 was less efficient in inducing Ab3 anti-p53 antibodies in either of the strains.

FIG. 4B shows that the sera contained a mixture of Ab3 p53-reactive antibodies of different IgG isotypes including IgG2b. Since the Ab1 antibodies used for immunization were either of the IgG1 (PAb-246, PAb-248) or IgG2a isotypes (PAb-421), it appears likely that the anti-p53 antibodies detected were indeed the Ab3 products of the idiotypic network, rather than merely remnants of the immunizing Ab1 antibodies. The finding that Ab3 anti-p53 were of T cell-dependent IgG isotypes implies the involvement of idiotype-specific T cells.

### Strain Differences in Ab3 Specificities

Because the two mouse strains differed in their Ab1 response to PAb-421, it was conceivable that they also might differ in their Ab3 response to this Ab1 antibody. The specificity of the Ab3 response was analyzed by probing test sera for antibodies to the C-terminal peptide epitope of p53 that is recognized by the inducing PAb-421 (amino acids 364-383 of SEQ ID NO:1). FIG. 5 shows that this p53 peptide was recognized by the Ab3 antibodies of the BALB/c mice.

In contrast, the C57BL/6 mice did not make antibodies to the C-terminal p53 peptide after Ab1 PAb-421 immunization, although they did make Ab3 anti-p53 antibodies of other specificities, as discussed above. Since the Ab2 response of the C57BL/6 mice to PAb-421 was cross-reactive to PAb-246, as shown in FIGS. 1 and 2, it is conceivable that their Ab3 response was preferentially directed to the central p53 domain. Thus, the specificity of the Ab3 antibodies in the two strains differed: the Ab3 induced in BALB/c mice recognized the p53 epitope of the inducing Ab1 PAb-421, while the Ab3 anti-p53 antibodies of C57BL/6 mice induced by Ab1 PAb-421 did not recognize the Ab1 epitope.

A comparison of the two mouse strains is summarized in Table 1.

TABLE 1
Comparison of the Response to Ab1 PAb-421 Immunization of
BALB/c and C57BL/6 Mice and of Spontaneous Immunity of
MRL/MpJ-Fas^{1 pr} Mice

| | | | MRL/MpJ- |
|---|---|---|---|
| | BALB/c | C57BL/6 | FdS^{1 pr} |
| proteinuria (>0.3 g/l) | + | − | + |
| nephritis | + | − | + |
| leukopenia (<4000/ml) | + | − | + |
| lymphopenia (<1200 ml) | + | − | + |
| skinrashes | + | − | − |
| Ab2 Anti-PAb-421 antibodies | + | + | + |
| anti-native DNA antibodies | + | − | + |
| (Crithidia) | | | |
| anti-single-stranded DNA (calf | + | − | + |
| thymus) | | | |
| cross-reactive Ab2 | − | + | not |
| | | | tested |
| Ab3 anti-p53 antibodies | + | + | + |
| Ab3to C-terminus of p53 | + | − | + |
| anti-histone antibodies | + | − | not |
| | | | tested |

### EXAMPLE 2

### Idiotypic Recognition of PAb-421 in a Mouse Strain that Develops SLE Spontaneously

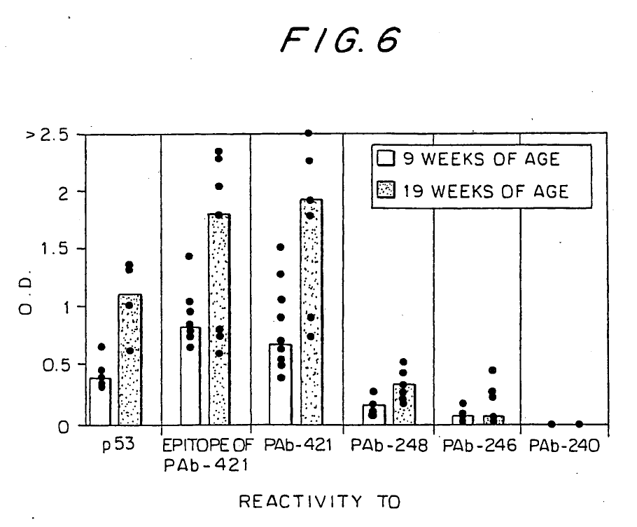

To learn whether an idiotypic recognition of the C-terminus of p53 might also play a role in the spontaneous development of SLE, MRL/MpJ-Fas^{lpr}, a strain that develops SLE spontaneously, was examined for the presence of antibodies to p53 and PAb-421. As can be seen in FIG. 6, these mice, 15 spontaneously on their way to SLE, make rising titers of antibodies to p53, to the p53 peptide epitope of PAb-421, and to PAb-421, but not to other anti-p53 antibodies (PAb-248, PAb-246, PAb-240). Thus, it appears that the same idiotypic network that was associated with SLE induction in the BALB/c mice was also operative in the spontaneous disease development of the MRL/MpJ-Fas^{lpr} mice.

As noted above, the immunization of BALB/c mice to two different Ab1 antibodies that each recognizes one of the two different DNA-binding domains of p53 resulted in idiotypic immune responses that included anti-DNA immunity. It is remarkable that the induced Ab2 anti-DNA in BALB/c mice apparently preserved the specificity of the DNA-binding domains of p53: immunization of Ab1 PAb-246 resulted in anti-DNA antibodies that bound specifically the pS3 p53-responsive element, and immunization to Ab1 PAb-421 resulted in non-specific DNA-binding detected in the Crithidia assay.

The immune response to Ab1 PAb-421 was associated with the development of murine lupus marked by a renal disorder (proteinuria and nephritis), hematologic disorders (leukopenia and lymphopenia), and an immunologic disorder (anti-DNA, anti-p53 and anti-histone antibodies). In contrast, the specific Ab2 anti-DNA response to the p53-responsive element of the BALB/c mice immunized to Ab1 PAb-246 did not lead to disease. Thus, the specificity of the anti-DNA immune response and its association with SLE disease is dictated by the specificity of the p53 DNA-binding domain.

A genetic influence on susceptibility was evident: BALB/c mice, but not C57BL/c mice, developed SLE by immunization to Ab1 PAb-421. Although both strains made Ab2 and Ab3 responses, the mice manifested major differences in their network specificities. The Ab2 response of the susceptible BALB/c mice was restricted to the Ab1 inducer, and was associated with reactivity to native DNA. The Ab2 response of the resistant C57BL/6 mice, in contrast, showed cross-reactivity to both PAb-421 and PAb-246, and was dominated by anti-PAb-421 antibodies that did not bind to DNA. The different Ab2 responses of the two strains resulted in different Ab3 anti-p53 responses: the BALB/c mince made antibodies that, like the Ab1 inducer PAb-421, bound to the C-terminal p53 peptide. C57BL/6 mice, however, did not make such antibodies.

It was particularly interesting that the MRL/MpJ-Fas^{lpr} mice, when they spontaneously developed SLE, made rising titers of antibodies with specificities that were associated with the development of SLE in the BALB/c strain: the MRL/MpJ-Fas^{lpr} mice spontaneously made anti-p53 antibodies, including antibodies to the C-terminal p53 peptide epitope of PAb-421, and anti-idiotypic antibodies to PAb-421. Since these mice did not produce significant antibody titers specific for other monoclonal antibodies, including other anti-p53 antibodies, this finding strongly suggests that the same idiotypic network drives disease development also in spontaneous SLE.

The following examples relate to the isolation of monoclonal antibodies that mimic the carboxy terminal domain of p53 and the unexpected discovery that such monoclonal antibodies specifically bind to damaged DNA.

### EXAMPLE 3

### Isolation of Monoclonal Antibodies that Mimic the Carboxy Terminal Domain of p53 and Bind Damaged DNA

To generate such antibodies, BALB/c mice were immunized with PAb-421 and selected hybridomas producing anti-idiotypic antibodies that bound PAb-421 and could also bind DNA were isolated.

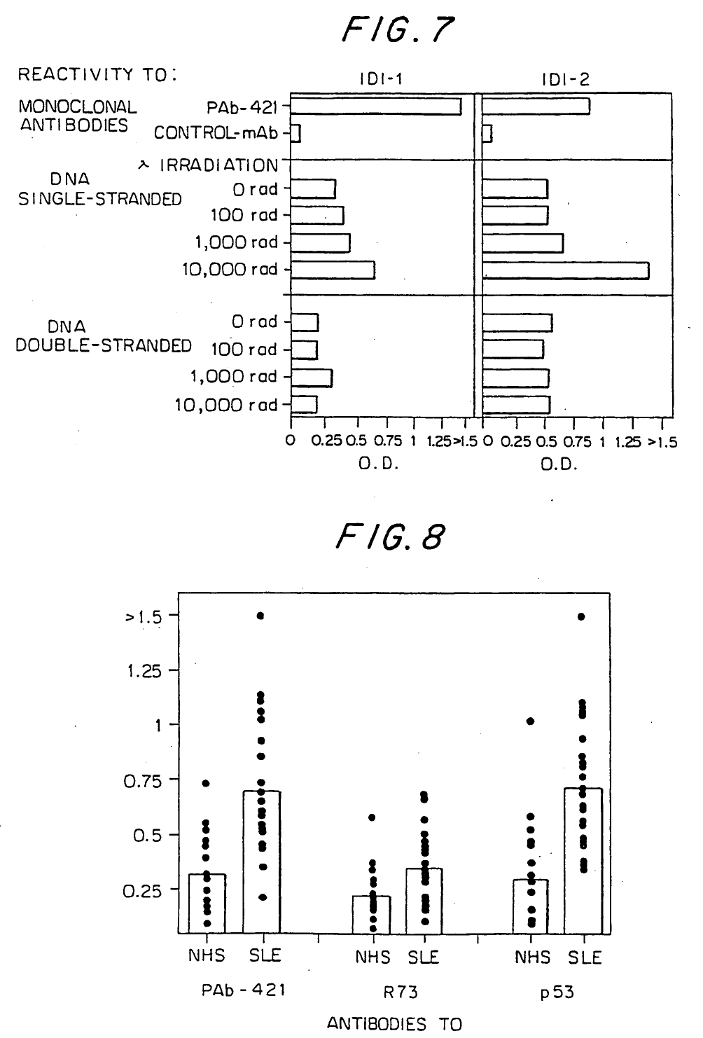

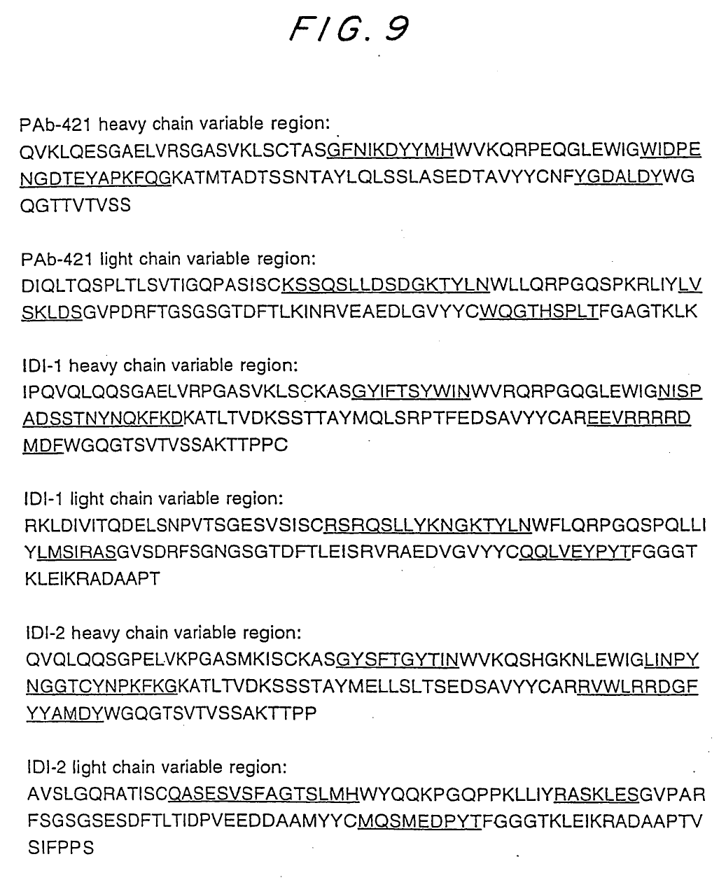

Two monoclonal antibodies, IDI-1 and IDI-2, could be isolated which had been selected for idiotypic binding to PAb-421 and their reactivities were determined. As shown in FIG. 7, both showed specific reactivity to PAb-421 and to DNA, single-stranded or double-stranded. No reactivity was found to mouse antibodies other than PAb-421, like R73. The binding of IDI-1 and somewhat less of IDI-2 to single-stranded DNA was markedly enhanced after gamma-irradiation of the DNA, depending on the radiation dose absorbed by the DNA. This is remarkable, since p53 also shows preferential binding to DNA damaged by gamma-irradiation (Reed et al, 1995), as well as to single-stranded DNA ends (Selivanova et al, 1996). Thus, these two novel monoclonal antibodies of the present invention appear to mimic the DNA-binding specificity of p53. Thus, such antibodies could serve as models of the critical regulatory conformation of the carboxy-terminal domain of p53.

### EXAMPLE 4

### The Monoclonal Antibodies IDI-1 and IDI-2 and Recognize a Similar DNA Motif

Preliminary studies were done to define the oligonucleotide motifs recognized by IDI-1 and IDI-2 and to test whether p53 itself might bind the same oligonucleotide. Single-stranded homooligomers (20-mers) of dG, dA, dC and dT were used. In a band-shift assay, it was shown that IDI-1, IDI-2 and p53 bound to the dG oligomer better than they did the others. Thus, dG is an important ligand for the anti-DNA monoclonal antibodies and for p53 itself. This further supports the presumptions that IDI-1 and IDI-2 mimic the carboxy terminal conformation of p53. In fact, the binding of the antibody to oligo dG is evidence that the antibody actually mimics the C-terminal domain of p53.

### EXAMPLE 5

### Oligo-dG Activates p53

To test whether the oligo-dG ligand is a functional regulator of p53, we investigated whether oligo-dG might activate p53 to recognize the p53 responsive element. Preliminary results using a gel shift assay and labeled p53 responsive element indicate that the activation of p53 is 10-fold more sensitive to oligo-dG than to each of the other three oligomers of dA, dT or dC. Thus, a ligand found to bind to both p53 and to IDI-1 and IDI-2 seems to serve as a functional regulator of p53.

Accordingly, the present invention is also directed to Ab2 monoclonal antibodies, such as IDI-1 and IDI-2, raised against monoclonal antibodies specific to the C-terminal domain of p53, which Ab2 antibodies have the unique capability of binding to damaged DNA. Thus, such antibodies can be used not only to find damaged DNA, but also as a model to study the regulatory conformation of the carboxy terminal domain of p53.

The following examples are directed to methods of treatment of SLE in accordance with the present invention.

### EXAMPLE 6

### Down-Regulation of SLE

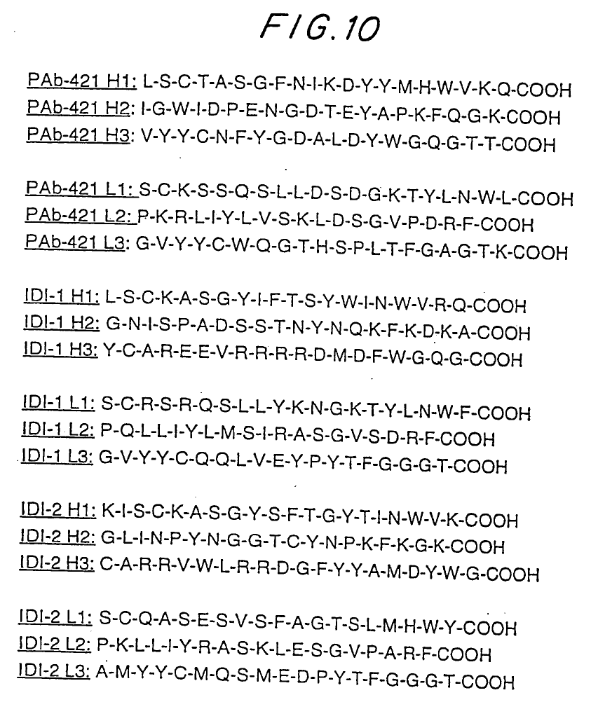

The above results indicated that a p53 immune network is associated with SLE. Thus, modulation of the p53 immune network should affect the course of SLE. A preliminary experiment was designed to see whether the spontaneous SLE development in MRL/MpJ-Fas^{lpr} mice might be influenced by administering to these mice peptides involved in the p53 network: the CDR peptides of the light and the heavy chain of PAb-421 depicted in FIG. 10 (administered as a mixture), or peptide 364-383 (residues 364-383 of SEQ ID NO:1) of the carboxy terminal domain of p53. The mice were intraperitoneally injected with 100 μg of the p53 peptide epitope of PAb-421 in PBS, or with 100 μg of peptides derived from variable region of PAb-421 in PBS at weeks 12,18, and 24 of age and followed for the development of SLE symptoms. As shown in Table 2, only 2 out of 5 mice (40%) treated with the p53 peptide epitope of PAb-421 (Table 2: 421-epitope) developed severe proteinuria at week 17 of age, compared to 6 out of 8 (75%) of the untreated control mice (Table 2: not treated) developing proteinuria. Treatment with the peptides derived from the variable region of PAb-421 protected all five mice from development of proteinuria (0%; Table 2: 421: CDR). At week 33 of age, 5 of 6 non-treated mice (83%, cf. Table 2) were dead, whereas none of the mice treated with either peptide had died (0% each; see Table 2).

Also carried out was T cell vaccination against immunity to PAb-421. MRL/MpJ mice were immunized intradermally with 30 μg PAb-421 in complete Freund's adjuvant, followed by a boost in incomplete Freund's adjuvant. Cells derived from the draining lymph nodes, which were enriched for T cells reactive to PAb-421 and the p53 epitope peptide of PAb-421, were activated with 2 μg per ml Concanavalin A. Twelve week old MRL/MpJ-Fas^{lpr} mice were then injected intraperitoneally with 2×10⁷ freshly activated and gamma-irradiated (2500 rad) cells per mouse. At 17 weeks of age, only 1 out of 8 treated mice (19%) developed severe proteinuria, while, in contrast, 6 out of 8 untreated control mice (75%) developed severe proteinuria. The results are shown in Table 2. 421-TCV vs. not treated.

TABLE 2
Prevention of Spontaneous SLE in MRL/MpJ-Fas^{1 pr} Mice

| | Not | | 421- | |
|---|---|---|---|---|
| | Treated | 421-CDR | Epitope | 421-TCV |
| heavy proteinuria, ³3 | 6/8 | 0/5 | 2/5 | 1/8* |
| g/l (+++) at week 17 of | (75%) | (0%) | (33%) | (13%) |
| age | | | | |
| death by week 33 of age | 5/6 | 0/6 | 0/6 | n.t |
| | (83%) | (0%) | (0%) | |

*significantly different from the non-treated control group, as assessed by Fisher's exact probability test

### EXAMPLE 7

### Anti-p53 and Anti-PAb-421 in Human SLE Patients

The finding of anti-p53 and anti-PAb-421 anti-idiotypic antibodies in the MRL/MpJ-Fas^{lpr} mouse model suggested that human SLE patients might also produce such antibodies. Randomly collected sera of 25 SLE patients and of 14 healthy humans were surveyed for antibodies to p53, to PAb-421, and to another mouse antibody, R73, that does not bind p53 (FIG. 8). The SLE sera produced significantly higher titers of anti-p53 and anti-PAb421 antibodies than the healthy control sera. In contrast, there was no significant difference between SLE patients and healthy subjects in their antibody titers to R73. Thus, a random group of SLE patients manifested the same p53 associated antibodies observed in the MRL/MpJ-Fas^{lpr} mice developing spontaneous SLE.

### EXAMPLE 8

### T Cell Vaccination

T cell lines specific for PAb-421, for peptides derived from the variable region of PAb-421, or for the p53 peptide epitope of PAb-421 or for IDI-1 or IDI-2 can be derived from peripheral blood lymphocytes (PBL) of SLE patients as described in Ota et al (1990). Briefly, PBL are separated from heparinized blood of SLE patients by Ficoll density gradient centrifuging. To obtain T cell lines, the PBL are repeatedly stimulated in tissue culture plates with 10 μg per ml of peptides derived from the variable regions of PAb-421, or with 10 μg per ml of the p53 peptide epitope of PAb-421 or IDI-1 or IDI-2 in the presence of a 5-20 fold greater concentration of gamma-irradiated (10,000 rad) autologous blood mononuclear cells, which are also derived by Ficoll density gradient centrifugation from heparinized blood. The stimulation can be performed in any suitable medium, such as RPMI 1640 medium, containing 10% autologous serum (or 10% pooled human AB serum), 1% HEPES buffer, 1% penicillin/streptomycin, and 1% glutamine. Three days after stimulation, 5 U/ml of recombinant human interleukin-2 are added. The line T cells can be cloned by limiting dilution, as described in Zhang et al (1993), if desired.

For vaccination, freshly activated T cells are attenuated, e.g., by gamma-irradiation (8000 rad), and 106-108 cells are injected subcutaneously in PBS as described in Zhang et al (1993), followed by several boosts with similar preparations.

### EXAMPLE 9

### Vaccination with Peptides Derived from the T Cell Receptor (TCR)

T cell clones specific for PAb-421, for peptides derived from the variable region of PAb-421, or for the p53 peptide epitope of PAb-421 or IDI-1 or IDI-2 were obtained from SLE patients as described in Example 8. After DNA extraction and polymerase chain reaction-based amplification of the cloned rearranged TCR-genes, the oligonucleotide sequence of the product of the polymerase chain reaction is determined (Maniatis et al, 1982). From the oligonucleotide sequence, the amino acid sequence of the TCR can be deduced.

Synthetic peptides derived from the TCR of PAb-421-specific T cells, or from the TCR of T cells specific for the C-terminus of p53, or from the TCR of IDI-1 or IDI-2 specific T cells, are then used for vaccination as described (Vandenbark et al, 1996). Briefly, SLE patients are inoculated intradermally with 0.1-0.5 mg of the said peptide, followed by several boosts.

### EXAMPLE 10

### Vaccination with Genes Encoding the TCR

Oligonucleotides corresponding to the rearranged T cell receptor genes of cloned T cells, specific for peptides derived from the variable region of PAb-421, or specific for the p53 peptide epitope of PAb-421 or IDI-1 or IDI-2, are obtained as described in Example 9. These oligonucleotides encoding the TCR, or parts thereof, are then cloned into suitable vectors (Sato et al, 1996), and patients are inoculated with 0.01-1 mg of the DNA construct as described (Waisman et al, 1996).

### EXAMPLE 11

### Anergy Induction by Intravenous or Subcutaneous Administration

For anergy induction, 0.1-100 mg of PAb-421, peptides derived from variable region of PAb-421, p53, or of the p53 peptide epitope of PAb-421 or IDI-1 or IDI-2, are administered to SLE patients subcutaneously in adjuvant as described (Elias et al, 1994), or intravenously in PBS as described (Gaur et al, 1992).

### EXAMPLE 12

### Anergy Induction by Enteric Administration (Oral Tolerance)

Oral tolerance can be induced by daily enteric | administration of 0.5-500 mg of p53, the p53 peptide epitope of PAb-421, PAb-421 or IDI-1 or IDI-2, or of peptides derived from the variable region of PAb-421 as described (Weiner et al, 1993).

To treat patients suffering from SLE, vaccine compositions can be prepared as follows:

A vaccine comprising activated T cell lines specific for PAb-421, for peptides derived from the variable regions of PAb-421, or for the p53 peptide epitope of PAb-421 or IDI-1 or IDI-2, together with a pharmaceutically acceptable carrier, can be administered through various routes known in the art, such as oral, intranasal, intravenous, subcutaneous, intramuscular, intraperitoneal, transdermal, or other known routes including the enteral route.

Alternatively, vaccines which are prepared from T cell clones specific for PAb-421, for peptides derived from the variable region of PAb-421, or for the p53 epitope of PAb-421 can be used together with a pharmaceutically acceptable carrier, can be administered through various routes known in the art, such as oral, intranasal, intravenous, subcutaneous, intramuscular, intraperitoneal, transdermal, or other known routes including the enteral route.

Oligonucleotides encoding the rearranged T cell receptor genes of cloned T cells specific for peptides derived from the variable region of PAb-421, or specific for the p53 peptide epitope of PAb-421, along with a pharmaceutically acceptable carrier, can be used to prepare vaccines to treat autoimmune diseases mediated by p53.

Compositions for intravenous or subcutaneous administration can be prepared from PAb-421, peptides derived from the variable region of PAb-421, p53, or of the p53 peptide epitope of PAb-421 and a pharmaceutically acceptable carrier. Alternatively, oral tolerance can be induced by daily enteric administration of p53, the p53 peptide epitope of PAb-421, PAb-421, or peptides derived from the variable region of PAb-421 and a pharmaceutically acceptable carrier for oral delivery.

The dosage of the peptide, oligonucleotide, etc. to be administered will depend on the type of compound used—an oligonucleotide, a peptide fragment, or a peptide, and upon the age, sex, weight, and condition of the recipient. The doses should not be so large as to cause adverse side effects such as unwanted cross-reactions, anaphylactic reaction, and the like.

Having now fully described this invention, it will be appreciated by those skilled in the art that the same can be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Aarden et al, “Immunology of DNA. III. Crithidia lucilie, a Simple Substrate for the Determination of anti-dsDNA with the Immunofluorescence Technique”, Ann. NY Acad. Sci. 254: 505-515 (1975)

Andrews et al, Spontaneous Murine Lupus-like Syndromes, Clinical and Immunopathological Manifestations in Several Strains”, J. Exp. Med. 148: 1198-1215 (1978)

Arai et al,“Immunologically Distinct p53 Molecules Generated by Alternative Splicing”, Mol. Cell. Biol. 6: 3232-9 (1986)

Baker et al, Suppression of Human Colorectal Carcinoma Cell Growth by Wild-Type p53”, Science 249: 912-915 (1990)

Ben-Nun et al, “Vaccination against Autoimmune Encephalomyelitis with T-lymphocyte Line Cells Reactive against Myelin Basic”, Nature 292: 60-61 (1981)

Cohen, I. R., “The Cognitive Paradigm and the Immunological Homunculus”, Immunol. Today 13: 490-494 (1992)

Cook et al, “Evidence for Allosteric Variants of Wild-type p53, a Tumour Suppressor Protein”, Br. J. Cancer 61: 548-552 (1990)

Dong et al, “Initiation of Autoimmunity to the p53 Tumor Suppressor Protein by Complexes of p53 and SV40 Large T Antigen”, J. Exp. Med. 179: 1243-1252 (1994)

el-Deiry et al, “Definition of a Consensus Binding Site for p53”, Nat. Genet. 1: 45-49 (1992) p Elias et al, “Peptide Therapy for Diabetes in NOD Mice”, Lancet 343: 704-706 (1994)

Erez-Alon et al, “Immunity to p53 Induced by an Idiotypic Network of anti-p53-antibodies: Generation of Sequence-specific anti-DNA Antibodies and Protection from Tumor Metastasis”, Cancer Res. 58: 5447-5452 (1998)

Eshhar et al, “Chimeric T cell receptor which incorporates the anti-tumour specificity of a monoclonal antibody with the cytolytic activity of T cells: a model system for immunotherapeutical approach”, Br. J. Cancer Suppl., 10: 27-29 (1990)

Gaur et al, “Amelioration of Autoimmune Encephalomyelitis by Myelin Basic Protein Synthetic Peptide-induced Anergy”, Science 258: 1491-1494 (1992)*

Gross et al, “Expression of immunoglobulin-T-cell receptor chimeric molecules as functional receptors with antibody-type specificity”, Proc. Natl. Acad. Sci. USA 86 (24): 10024-10028 (1989)

Harlow et al, Antibodies: A Laboratory Manual, Cold Spring Harbor Laboratory (Cold Spring, 1988), p. 626

Harris et al, “Molecular Basis for Heterogeneity of the Human p53 protein”, Mol. Cell. Biol. 6: 4650-4656 (1986)

Higgins et al, “Suppression of Experimental Autoimmune Encephalomyelitis by Oral Administration of Myelin Basic Protein and its Fragments”, J. Immunol. 140: 440-445

Hollstein et al, “p53 Mutations in Human Cancers”, Science 253: 49-53 (1991)

Howell et al, “Vaccination against Experimental Allergic Encephalomyelitis with T Cell Receptor Peptides”, Science 246:688-670, 1989

Kastan et al, “Participation of p53 Protein in the Cellular Response to DNA Damage”, Cancer Res. 51: 6304-6311 (1991)

Kelley et al, “Interaction of Mutant lpr Gene with Background Strain Influences Renal Disease”, Clin. Immunol. Immunopathol. 37: 220-229 (1985)

Kuerbitz et al, “Wild-type p53 Is a Cell Cycle Checkpoint Determinant Following Irradiation”, Proc. Natl. Acad. Sci. USA 89: 7491-7495 (1992)

Lee et al, “p53 and Its 14 kDa C-terminal Domain Recognize Primary DNA Damage in the Form of Insertion/Deletion Mismatches”, Cell 81: 1013-1020 (1995)

Linzer et al, “Characterization of a 54K Dalton Cellular SV40 Tumor Antigen Present in SV40-transformed Cells and Uninfected Embryonal Carcinoma Cells”, Cell 17: 43-52 (1979)

Maniatis et al, Molecular Cloning: A Laboratory Manual, Cold Spring Harbor Laboratory Press (Cold Spring Harbor, N.Y., 1982)

Mendlovic et al, “Induction of a Systemic Lupus Erythematosus-like Disease in Mice by a Common Human Anti-DNA Idiotype”, Proc. Natl. Acad. Sci. USA 85: 22602264 (1988)

Moens et al, In Vivo Expression of a single Viral DNA-binding Protein Generates Systemic Lupus Erythematosus-related Autoimmunity to Double-stranded DNA and Histones”, Proc. Natl. Acad. Sci. USA 92: 12393-12397 (1995)

Ota et al, “T-cell Recognition of an Immunodominant Myelin Basic Protein Epitope in Multiple Sclerosis”, Nature 346: 183-187 (1990)

Pisetsky, D. S., “The Immunologic Properties of DNA”, J. Immunol. 156: 421-423 (1996)

Radic et al, “Genetic and Structural Evidence for Antigen Selection of Anti-DNA Antibodies”, Annu. Rev. Immunol. 12: 487-520 (1994).

Reed et al, “The C-terminal Domain of p53 Recognizes DNA Damaged by Ionizing Radiation”, Proc. Natl. Acad. Sci. USA 92: 9455-9459 (1995)

Ruiz et al, “Immunity to Mutant p53 and Tumor Rejection Induced by Idiotypic Immunization”, Nat. Med. 4: 710-712 (1998)

Sato et al, “Immunostimulatory DNA Sequences Necessary for Effective Intradermal Gene Immunization”, Science 273: 352-354 (1996)

Selivanova et al, “The Single-stranded DNA End Binding Site of p53 Coincides with the C-terminal Regulatory Domain”, Nucleic Acids Research 24: 3560-3567 (1996)

Shohat-Foord et al, “A DNA Binding Domain Is Contained in the C-terminus of Wild Type p53 Protein”, Nucleic Acids Res. 19: 5191-5198 (1991)

Vandenbark et al, “Immunization with a Synthetic T-cell Receptor V-region Peptide Protects against Experimental Autoimmune Encephalomyelitis”, Nature 341: 541-544 (1989)

Vandenbark et al, “Treatment of Multiple Sclerosis with T-cell Receptor Peptides: Results of a Double-blind Pilot Trial”, Nat. Med. 2: 1109-1115 (1996)

Waisman et al, “Suppressive Vaccination with DNA Encoding a Variable Region Gene of the T-cell Receptor Prevents Autoimmune Encephalomyelitis and Activates ThM Immunity”, Nat. Med. 2: 899-905 (1996)

Ward et al, “Natural Antibodies that React with V-region Peptide Epitopes of DNA-binding Antibodies Are Made by Mice with Systemic Lupus Erythematosus as Disease Develops”, Immunology 92: 354-361 (1997)

Watanabe-Fukunaga et al, “Lymphoproliferation Disorder in Mice Explained by Defects in Fas Antigen that Mediates Apoptosis”, Nature 356: 314-317 (1992)

Weiner et al, “Double-blind Pilot Trial of Oral Tolerization with Myelin Antigens in Multiple Sclerosis”, Science 259: 1321-1324 (1993)

Wolkowicz et al, “The DNA Binding Activity of Wild Type p53 Is Modulated by Blocking its Various Antigenic Epitopes”, Oncogene 10: 1167-1174 (1995)

Yewdell et al, “Monoclonal Antibody Analysis of p53 Expression in Normal and Transformed Cells”, J. Virol. 59: 444-452 (1986)

Yonish-Rouach et al, Wild-type p63 Induces Apoptosis of Myeloid Leukaemic Cells that Is Inhibited by Interleukin-6”, Nature 352: 345-347 (1991)

Zhang et al, “MHC-restricted Depletion of Human Myelin Basic Protein-reactive T Cells by T Cell Vaccination”, Science 261: 1451-1454 (1993)

1. A method for treating systemic lupus erythematosus (SLE) in a human with SLE or suffering from the immune reaction that causes SLE, comprising down-regulating the autoimmune response in the human to the C-terminal DNA-binding domain of the human p53 protein (p53).

2. A method in accordance with claim 1 for treating SLE in a human patient suffering from SLE.

3. A method in accordance with claim 1, wherein said down-regulating step comprises administering to the human, in a manner which suppresses the autoimmune response to the C-terminal DNA-binding domain of p53, an active principle which is a polypeptide selected from the group consisting of:
(a) a portion of human p53 which includes the C-terminal DNA-binding domain thereof;
(b) the antigen binding domain of an antibody (Ab1) or T cell (Tc1) which manifests specificity against the C-terminal DNA-binding domain of human p53;
(c) the antigen binding domain of an antibody (Ab2) which manifests specificity against Ab1 or the TCR of Tc1, or the TCR of a T cell (Tc2) which manifests specificity against the antigen binding domain of Ab1 or the TCR of Tc1; and
(d) a fraction of (a) or (c) which can be used as an immunogen to raise antibodies, the anti-idiotypic antibodies of which bind to DNA, or a fraction of (b) which can be used as an immunogen to raise antibodies which bind to DNA.

4. A method in accordance with claim 3, wherein said active principle is a portion of human p53 which includes the C-terminal DNA-binding domain thereof.

5. A method in accordance with claim 3, wherein said active principle is a fraction of (a) or (c) which can be used as an immunogen to raise antibodies, the anti-idiotypic antibodies of which bind to DNA, or a fraction of (b) which can be used as an immunogen to raise antibodies which bind to DNA.

6. A method in accordance with claim 1, wherein said human being treated is one who is positive in a diagnostic test comprising testing said human for the presence of antibodies (Ab1) against the C-terminal DNA-binding domain of the human p53 protein or antibodies (Ab2) against the Ab1 antibodies, whereby a result indicating the positive presence of either said Ab1 or Ab2 antibodies is a positive result.

7. A method in accordance with claim 1, wherein said human being treated is one who is positive in a diagnostic test comprising testing said human for the presence of antibodies or T cells that immunoreact with the C-terminal DNA-binding domain of the human p53 protein (p53) or for antibodies or T-cells that immunoreact with antibodies or T-cells that are specific to the C-terminal DNA-binding domain of human p53, whereby a result indicating the positive presence of such antibodies or T cells is a positive result.

8. A method for diagnosing the presence or incipience of systemic lupus erythematosus (SLE) in a patient, comprising testing said patient for the presence of antibodies (Ab1) against the C-terminal DNA-binding domain of the human p53 protein or antibodies (Ab2) against the Ab1 antibodies, whereby a result indicating the positive presence of either said Ab1 or Ab2 antibodies indicates a high probability of the presence of incipience of SLE.

9. A method for diagnosing for the presence or incipience of systemic lupus erythematosus (SLE) in a patient, comprising testing said patient for the presence of antibodies or T cells that immunoreact with the C-terminal DNA-binding domain of the human p53 protein (p53) or for antibodies or T cells that immunoreact with antibodies or T cells that are specific to the C-terminal DNA-binding domain of p53, whereby a result indicating the positive presence of such antibodies or T cells indicates a high probability of the presence or incipience of SLE.

10. A method in accordance with claim 3, wherein said active principle is administered by administering DNA encoding the protein or polypeptide of the active principle a manner in which said DNA is caused to express said active principle in vivo.

11. A method in accordance with claim 3, wherein said active principle is the antigen binding domain of an antibody (Ab1) or T cell (Tc1) which manifests specificity against the C-terminal DNA-binding domain of p53.

12. A method in accordance with claim 11, wherein said antibody Ab1 is PAb-421.

13. A method in accordance with claim 11, wherein said active principle is the PAb-421 monoclonal antibody.

14. A method in accordance with claim 3, wherein said active principle is the antigen binding domain of an antibody (Ab2) which manifests specificity against Ab1 or the TCR of Tc1, or the TCR of a T cell (Tc2) which manifests specificity against the antigen binding domain of Ab1 or the TCR of Tc1.

15. A method in accordance with claim 14, wherein said active principle is the IDI-1 or IDI-2 monoclonal antibody.

16. A method in accordance with claim 15, wherein said fraction is a peptide including the CDR of the heavy or light chain of antibody Ab1 or the CDR of the heavy or light change of antibody Ab2.

17. A method in accordance with claim 16, wherein said peptide is one which includes a sequence selected from the group consisting of residues 26-35, 50-66 and 99-105 of SEQ ID NO:2, residues 24-39, 55-61 and 94-102 of SEQ ID NO:3, residues 28-37, 52-68 and 101-111 of SEQ ID NO: 4, residues, 2742, 58-64 and 97-105 of SEQ ID NO:5, residues 26-35, 50-66 and 99-113 of SEQ ID NO:6, and residues 13-27, 43-49 and 82-90 of SEQ ID NO:7.

18. A method in accordance with claim 1, wherein said down-regulating step comprises administering to a human, in a manner that suppresses the autoimmune response to the C-terminal DNA-binding domain of the p53 protein, an active principle selected from the group consisting of:
(a) activated human T cells which manifest specificity for the C-terminal domain of human p53, human T cells which manifest specificity for a monoclonal antibody specific to the C-terminal DNA-binding domain of the human p53 protein (Ab1), or human T cells which manifest specificity for monoclonal antibodies which are specific for the Ab1 antibody (Ab2);
(b) said human T cells of (a) which have been attenuated by gamma- or UV irradiation;
(c) said human T cells of (a) which have been subjected to pressure treatment by means of hydrostatic pressure, treatment with chemical cross-linking agent and/or treatment with a cytoskeletal cross-linking agent;
(d) fragments of, or surface proteins shed from, the T cells of (a), (b) or (c); and
(e) a peptide comprising the variable region of the T cell receptor of the T cells of (a).

19. A method in accordance with claim 18, wherein said T cell product is said human T cells of (a).

20. A method in accordance with claim 18, wherein said T cell product is said attenuated T cells of (b) or said treated T cells of (c).

21. A method in accordance with claim 18, wherein said T cell product is said fragments or shed proteins of (d).

22. A method in accordance with claim 18, wherein said T cell product is said peptide (e).

23. A method in accordance with claim 18, wherein said human T cells are autologous T cells from the patient to be treated or semi-allogeneic T cells from a donor sharing at least one HLA class II molecule with said patient.

24. A method in accordance with claim 18, wherein said human T cells of (a) are ones which manifest specificity to the C-terminal domain of human p53.

25. A method in accordance with claim 18, wherein said human T cells of (a) are ones which manifest specificity to said Ab1 antibodies.

26. A method in accordance with claim 18, wherein said human T cells of (a) are ones which manifest specificity to said Ab2 antibodies.

27. A method in accordance with claim 18, wherein said T cell product is administered in a manner which causes an immune response to be mounted against said T cell product.


SEQUENCE LISTING (10 sequences)

SEQ ID NO: 1 - protein, 390 aa, Mus sp.
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 2 - protein, 116 aa, Mus sp.
  BINDING (26)..(35): Complementary Determining Region
QVKLQESGAELVRSGASVKLSCTASGFNIKDYYMHWVKQRPEQGLEWIGWIDPENGDTEYAPKFQGKATMTADTSSNTAYLQLSSLASEDTAVYYCNFYGDALDYWGQGTTVTVSS

SEQ ID NO: 3 - protein, 110 aa, Mus sp.
  BINDING (24)..(39): Complementary Determining Region
DIQLTQSPLTLSVTIGQPASISCKSSQSLLDSDGKTYLNWLLQRPGQSPKRLIYLVSKLDSGVPDRFTGSGSGTDFTLKINRVEAEDLGVYYCWQGTHSPLTFGAGTKLK

SEQ ID NO: 4 - protein, 129 aa, Mus sp.
  BINDING (28)..(37): Complementary Determining Region
IPQVQLQQSGAELVRPGASVKLSCKASGYIFTSYWINWVRQRPGQGLEWIGNISPADSSTNYNQKFKDKATLTVDKSSTTAYMQLSRPTFEDSAVYYCAREEVRRRRDMDFWGQGTSVTVSSAKTTPPC

SEQ ID NO: 5 - protein, 122 aa, Mus sp.
  BINDING (27)..(42): Complementary Determining Region
RKLDIVITQDELSNPVTSGESVSISCRSRQSLLYKNGKTYLNWFLQRPGQSPQLLIYLMSIRASGVSDRFSGNGSGTDFTLEISRVRAEDVGVYYCQQLVEYPYTFGGGTKLEIKRADAAPT

SEQ ID NO: 6 - protein, 130 aa, Mus sp.
  BINDING (26)..(35): Complementary Determining Region
QVQLQQSGPELVKPGASMKISCKASGYSFTGYTINWVKQSHGKNLEWIGLINPYNGGTCYNPKFKGKATLTVDKSSSTAYMELLSLTSEDSAVYYCARRVWLRRDGFYYAMDYWGQGTSVTVSSAKTTPP

SEQ ID NO: 7 - protein, 114 aa, Mus sp.
  BINDING (13)..(27): Complementary Determining Region
AVSLGQRATISCQASESVSFAGTSLMHWYQQKPGQPPKLLIYRASKLESGVPARFSGSGSESDFTLTIDPVEEDDAAMYYCMQSMEDPYTFGGGTKLEIKRADAAPTVSIFPPS

SEQ ID NO: 8 - DNA, 26 bp, Homo sapiens
tcgagaggcatgtctaggcatgtctc

SEQ ID NO: 9 - DNA, 1307 bp, Homo sapiens
  CDS (126)..(1304)
accgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcgactcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 10 - protein, 393 aa, Homo sapiens [sequence omitted]